(19) United States
(12) Patent Application Publication
(10) Pub. No.: US 2025/0302816 A1
(43) Pub. Date: Oct. 2, 2025

(54) DISRUPTION OF EPITHELIAL BARRIERS FOR MOLECULAR DELIVERY ENHANCEMENT OR EXTRACTION OF EXTRACELLULAR FLUIDS

(71) Applicants: The University of Nottingham, Nottingham, Nottinghamshire (GB); Nottingham University Hospitals NHS Trust, Nottingham, Nottinghamshire (GB)

(72) Inventors: Kevin WEBB, Nottingham (GB); Emilia MORADI, Nottingham (GB); Alexander FOSS, Nottingham (GB)

(21) Appl. No.: 18/881,217

(22) PCT Filed: Jul. 5, 2023
(86) PCT No.: PCT/GB2023/051782

§ 371 Date: Jan. 3, 2025

(30) Foreign Application Priority Data
Jul. 5, 2022 (GB) 2209867.7

(51) Int. Cl. A61K 31/47; A61K 9/00; A61K 31/201; A61K 31/426; A61K 31/4412; A61K 38/17; A61K 39/00; A61K 39/395

(52) U.S. Cl. CPC A61K 31/47; A61K 9/0048; A61K 31/201; A61K 31/426; A61K 31/4412; A61K 38/179; A61K 39/3955; A61K 2039/505

(57) ABSTRACT

The invention provides a method of delivering a payload molecule across an epithelial tissue barrier, the method comprising: applying the payload molecule to the epithelial tissue barrier, and additionally applying an agent to the epithelial tissue barrier. The agent is (i) a microbial quorum sensing signalling molecule (microbial QSSM) or a derivative or variant thereof, which is capable of disrupting the epithelial tissue barrier function, or (ii) a carboxylic acid compound, or a pharmaceutically acceptable salt, hydrate or solvate thereof.

The present invention relates to a method of disrupting the epithelial tissue barrier, such as for delivering a payload molecule across an epithelial tissue barrier, lowering epithelial electrical resistance, or for extraction of extracellular fluids though an epithelial tissue barrier such as skin. The extracellular fluid may be used for analysis, such as glucose concentration or biomarker analysis. The lowered epithelial electrical resistance may be exploited to improve the sensitivity and signal to noise ratio for electrophysiological measurements. The invention further relates to associated compositions, uses and treatments.

Treatment of serious disease by modern precision medicine remains complicated by the inability of macromolecular therapies (non-limiting examples including immunotherapies, recombinant proteins and the like) to reach their site of action across tissue barriers (e.g. skin, lung, gut, ocular surface, nasal, oral, vaginal). Many of the most promising new therapeutics are large proteins, such as monoclonal antibodies and recombinant proteins. The rapid growth in macromolecular, protein-based drugs has unfortunately not been matched by developments in effective delivery systems for these novel therapeutics. Most proteins are administered parenterally due to their high molecular weight, causing an inability to penetrate epithelial barriers, and due to their inability to tolerate the acidic environment of the gastrointestinal tract. Disadvantages associated with the parenteral route, including patient discomfort and high cost, have stimulated the field to investigate non-invasive methods for administering macromolecular therapeutics.

Epithelia form barriers to protect tissues against ingested substances and pathogens. Their barrier properties arise from a set of proteins (tight junctions, adherens junctions and desmosomes) which restrict the intercellular space between cells. Junctional complexes link the cells, and it is the tight junctions that provide the barrier to free passage of molecules in the extracellular space. Tissue barriers are formidable obstacles in drug delivery, since drugs need to cross these barriers to reach their site of action and exert their therapeutic effects. The low permeability of macromolecular therapies across tissue barriers inhibits the efficient treatment of costly and prevalent diseases, including arthritis, macular degeneration and cancer. The delivery of protein therapeutics across various epithelial barriers, including gastrointestinal, respiratory, nasal, and buccal epithelia, remains a challenge for both the pharmaceutical industry and the clinical and academic communities.

Previous studies have shown that junctional integrity of epithelial cells can be modulated by microbial quorum sensing signalling molecules (see: Vikstrom, E., et al., Exp Cell Res, 2009. 315 (2): p. 313-26; Vikstrom, E., et al., FEBS Lett, 2006. 580 (30): p. 6921-8; Rejman, J., et al., Human Gene Therapy, 2007. 18 (7): p. 642-652).

In order to communicate, bacteria secrete extracellular signalling molecules called autoinducers. This cell-to-cell communication system is called “quorum sensing”, which assists bacteria to estimate their population, to monitor the environment, and to alter gene expression and consequently their behaviours such virulence factor production and biofilm formation (see: Whiteley, M. et al Nature, 2017. 551 (7680): p. 313-320; Papenfort, K. and B. L. Bassler, Nature Reviews Microbiology, 2016. 14 (9): p. 576-588). The opportunistic human pathogen Pseudomonas aeruginosa uses acyl-homoserine lactone (AHL) quorum sensing molecule to control and activate its gene expression.

Different strategies have been investigated to improve the permeability of macromolecular therapies into and across tissue barriers. These include the use of absorption enhancers, mucoadhesive excipients and attempts to exploit epithelial transcytosis. Absorption (or permeation/permeability) enhancers are a class of excipients which to increase drug permeability across both epithelial and endothelial cell layers leading to increased drug delivery to the systemic circulation.

Tight junctions can be disrupted by many agents, including toxins, cytokines, growth factor, surfactants, calcium chelators, polymeric vehicles such as chitosan, and some peptides. Whilst disruption of tight junctions can enhance drug delivery, a permanent dysfunction in tissue barrier function often results from a disorganisation of the tight junctions, which is unfavourable to their general use in clinical medicine as drug delivery enhancers. An ideal tight junction modulator must disrupt the barrier properties of epithelial layers both in a timely manner, and also in a reversible fashion.

There is need to overcome at least the above problems and to provide alternative, and preferably improved, methods and compositions for disruption of epithelial barriers, such as for delivery of molecules across epithelial barriers.

### SUMMARy OF THE INVENTION

The present invention has determined that a series of agents are able to enhance paracellular permeability for payload molecules. This series of agents acts to dislocate the ZO-1 proteins and, without being bound by theory, they may potentially act to impair barrier function through induction of matrix metalloprotease (MMP) secretion.

The effectiveness of these agents has been evidenced in relation to paracellular permeability for 4 kDa FITC-dextran (FD4) across polarized Calu-3, ARPE-19 and Caco-2 cell layers, well-known in-vitro models of human airway, gut and retinal pigment epithelium. Surprisingly, macromolecular agents (aflibercept, bevacizumab, mepolizumab: ˜150 kDa) have also been shown to be effectively delivered across epithelial barriers by using the present invention. It would not have been expected that molecules of this size could be delivered. The present invention advantageously provides a versatile approach that can be used with a wide range of payload molecules. To be able to effectively transport drugs having a range of sizes by penetrating the epithelial barrier is technically significant and offers significant advantages over intravenous administration.

Advantageously, this series of agents according to the invention has been found to cause the modulation of the tight junctions between barrier-forming cells in a reversible manner, with there being a return to baseline TEER and barrier function to macromolecules. This is both a beneficial and unexpected effect for a series of agents described herein. Having a resolution phase, i.e. repair, is important, because to be useful in practical and therapeutic terms the disruption of epithelial barriers needs to be temporary rather than there being permanent disruption or damage.

Thus, the epithelial barrier can be “opened”, allowing the payload molecule, e.g. therapeutic or prophylactic agent, to be delivered to the desired target across epithelial barriers, and then the epithelial barrier reverts to its normal configuration, i.e. it is “closed”.

As well as identifying this series of agents and using human cell lines to evidence their ability to reversibly disrupt the epithelial tissue barrier function, exemplary compounds from the series have successfully been further tested, both ex vivo and in vivo, to support the practical application of these agents.

In this regard, N-(3-oxododecanoyl) homoserine lactone (3OC12-HSL) and 2-n-heptyl-3-hydroxy-4 (1H)-quinolone (C7 PQS), which are exemplary of two distinct classes of microbial quorum sensing molecule within the claimed series, were confirmed as being effective at delivering drugs across the epithelial layers in in-vitro and in-vivo models, whilst also having been shown in experimental studies to have a resolution phase. The experimental studies showed that by including an agent according to the invention, drugs could be delivered successfully using eyedrops which normally would need to be injected, and furthermore no signs of toxicity were observed.

Therefore, the agents according to the invention offer a new and useful route for delivery of payload molecules, such as therapeutic or prophylactic agents, to subjects such as a human patient or a domestic animal or livestock.

The agents found by the present inventors to be effective in enhancing paracellular permeability for payload molecules whilst also having a resolution phase, i.e. such that the disruption of the epithelial barrier is temporary, have certain structural features in common. They all have an alkane “tail” portion comprising an R′ group which is a C1-12 alkyl group, that may be unsubstituted or substituted, wherein any substituent groups that are present are independently selected from hydroxyl, halogen and NR″2, where each R″ is independently selected from hydrogen and methyl. This “tail” therefore is saturated, only containing single bonds, and so is flexible. The agents also have either (i) a heterocyclic ring portion or (ii) a carboxylic acid plus alkene portion; this therefore provides a less flexible/more rigid portion which includes at least one heteroatom.

The invention provides, in a first aspect, a method of delivering a payload molecule across an epithelial tissue barrier, the method comprising:
applying the payload molecule to the epithelial tissue barrier, and
additionally applying an agent to the epithelial tissue barrier,
   wherein the agent is selected from:
(i) a microbial quorum sensing signalling molecule (microbial QSSM) or a derivative or variant thereof, which is capable of disrupting the epithelial tissue barrier function, and which is a compound that comprises a heterocyclic ring portion and an alkane portion comprising an R′ group which is a C1-12 alkyl group, that may be unsubstituted or substituted, wherein any substituent groups that are present are independently selected from hydroxyl, halogen and NR″2, where each R″ is independently selected from hydrogen and methyl; or
(ii) a carboxylic acid compound of Formula (I), or a pharmaceutically acceptable salt, hydrate or solvate thereof:

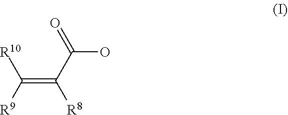

wherein
R⁸ is H or R′; R⁹ is H or R′; and R¹⁰ is H or R′, provided that at least one R′ group is present; wherein each R′ is independently selected from a C1-12 alkyl group, that may be unsubstituted or substituted, wherein any substituent groups that are present are independently selected from hydroxyl, halogen and NR″₂, where each R″ is independently selected from hydrogen and methyl.

The agents capable of disrupting the epithelial tissue barrier according to the invention may be microbial quorum sensing signalling molecules secreted by bacteria or may be other agents produced by bacteria. In this regard, the carboxylic acid compound of Formula (I) may be produced by bacteria, for example cis-2-decenoic acid is produced by bacteria. The agents may also be synthetic (i.e. non-natural). In particular, the agent may be synthetically/artificially produced, and the agent may or may not be produced or extracted from a microbe.

The invention further provides, in a second aspect, a pharmaceutically acceptable composition comprising (a) an agent capable of disrupting the epithelial tissue barrier, and optionally (b) a payload molecule; wherein the agent capable of disrupting the epithelial tissue barrier is as defined in the first aspect.

Also provided, in a third aspect, is the composition of the second aspect for use as a medicament, wherein the composition comprises a therapeutic or prophylactic payload molecule.

In a fourth aspect, there is provided the composition of the second aspect for use in a method of treatment or prevention of an eye disorder, a respiratory disorder, a gastrointestinal disorder, a reproductive-tract disorder, an auto-immune disorder, a mucous membrane disorder, a brain disorder, a microbial or parasitic infection, cancer, or a skin disorder, in a subject.

This therefore further provides a method of treatment or prevention of an eye disorder, a respiratory disorder, an auto-immune disorder, a gastrointestinal disorder, a reproductive-tract disorder, a mucous membrane disorder, a brain disorder, an infection, cancer, or a skin disorder,

the method comprising administering to a subject the composition of the second aspect, wherein the composition comprises a therapeutic or prophylactic payload molecule.

The treatment or prevention of a medical condition can involve direct delivery of the therapeutic or prophylactic payload molecule to the target tissue, or may involve indirect delivery of the therapeutic or prophylactic payload molecule to the target tissue. In particular, both local administration and systemic administration are foreseen. Thus, in one embodiment, for example, the therapeutic or prophylactic payload molecule may be directly delivered to the eye to treat or prevent an eye disorder, or may be directly delivered to the skin to treat or prevent a skin disorder. In another embodiment, the therapeutic or prophylactic payload molecule may be delivered indirectly by being delivered to the bloodstream, or may be delivered indirectly by being delivered to a location adjacent to the target tissue or forming a wider part of the body which comprises the target tissue. For example, the therapeutic or prophylactic payload molecule may be applied to the nail bed or another location adjacent to the nail such that it will then be delivered via the bloodstream to a location under the fingernail or toenail, e.g to treat a fungal infection. As another example, the therapeutic or prophylactic payload molecule may be applied to the nose such that it will then be delivered to the brain.

In a fifth aspect, there is provided a product comprising a composition of the second aspect, wherein the product is:
a) an eye drop dispenser, eye wash device or contact lens; and/or
b) an aspirator, inhaler, nebuliser, or vape device; and/or
c) a controlled release tablet or capsule suitable for oral administration; and/or
d) a transdermal patch or gel; and/or
e) a vaccine, wherein the vaccine further comprising an antigen or a nucleic acid (such as a viral vector) suitable for expression of an antigen.

In a sixth aspect, there is provided a kit comprising:
(a) an agent capable of disrupting the epithelial tissue barrier, and
(b) a payload molecule;

wherein the agent capable of disrupting the epithelial tissue barrier is as defined in the first aspect.

The invention also provides, in a seventh aspect, the use of an agent capable of disrupting the epithelial tissue barrier to facilitate penetration of a payload molecule through an epithelial tissue barrier, wherein the agent capable of disrupting the epithelial tissue barrier is as defined in the first aspect.

The invention also provides, in an eighth aspect, the use of an agent capable of disrupting the epithelial tissue barrier to facilitate extracellular fluid extraction from a subject, wherein the use comprises the application of the agent capable of disrupting the epithelial tissue barrier to an epithelial tissue barrier and the extraction of extracellular fluid through the epithelial tissue barrier,

wherein the agent capable of disrupting the epithelial tissue barrier is as defined in the first aspect.

In addition, in a ninth aspect there is provided a method for extracellular fluid extraction from a subject, the method comprising:
application of an agent capable of disrupting the epithelial tissue barrier to an epithelial tissue barrier of the subject; and
extraction of extracellular fluid through the epithelial tissue barrier;
   wherein the agent capable of disrupting the epithelial tissue barrier is as defined in the first aspect.

The invention herein advantageously provides agents that can be used for the disruption of epithelial barriers, allowing the passage of payload molecules, including macromolecules. The payload molecule may be applied to the epithelial tissue (for example respiratory tissue, skin surface, GI tract) that is the intended target (i.e. local administration), However, the payload molecule may also be applied to tissue that is not the intended target and the molecule is then further transported to the intended target, e.g. via the bloodstream (systemic administration). Thus the payload molecule can be applied to a location adjacent to the target tissue or forming a wider part of the body which comprises the target tissue. The payload molecule can be delivered to a distal location via the bloodstream from the point of introduction.

The agents according to the invention have been shown to be effective in Transwell macromolecular transport assays. Selected agents have then been further tested by ex vivo studies which supported the migration assay data in showing effectiveness for the agents to reversibly break tight junctions, for example in relation to the eyes and the skin. Yet further tests have confirmed effectiveness for agents according to the invention in in vivo studies.

In this regard, it is advantageously shown herein that agents according to the invention, such as 3OC12-HSL, can be used to enhance the transport of macromolecular therapeutics, such as bevacizumab (Avastin®), aflibercept (Eylea®), mepolizumab and doxorubicin (a widely used first-line chemotherapeutic) across in-vitro and ex-vivo epithelial barrier tissue models. Bevacizumab (MW 149 kDa) is a recombinant humanized monoclonal IgG1 antibody which binds to human vascular endothelial growth factor (VEGF), used to inhibit the aberrant growth of blood vessels in several cancers and in treating age-related macular degeneration (AMD). Aflibercept (MW 115 kDa) is a recombinant fusion protein which incorporates VEGF-binding portions from the extracellular domains of human VEGF receptors 1 and 2, and the Fc portion of human IgG1. Bevacizumab and aflibercept are commonly injected to reach their site of action in clinical practice, at significant expense. Doxorubicin (MW 543.52 Da) is an anthracycline antibiotic which binds to nucleic acids by specific intercalation of the planar anthracycline nucleus with the DNA double helix, disrupting DNA synthesis within rapidly-dividing cells.

Advantageously, the ability to temporarily disrupt the epithelial barrier for delivery of molecules can be used for a wide range of applications across a broad range of fields, such as the delivery of vaccines, including nucleic acid- and polypeptide-based vaccines, for example for coronavirus, through the skin; the use in tattoo pigmentation; lung or respiratory tract delivery of therapeutics; gastrointestinal or oral delivery of therapeutics; and ocular delivery.

Such delivery of molecules can be achieved via a number of application routes, such as gels, ointments, creams, patches, and aerosols (i.e. to the lungs).

It is beneficial that payload molecules such as drugs can be delivered without the need for injections; this improves the quality of life for patients and improves comfort, reduces the need for hospital visits, and reduces cost.

The present invention also recognises that the ability to disrupt the epithelial barrier for delivery of molecules can also be used in reverse for extraction of interstitial fluids between cells, which can contain biomarkers, vesicles or particles for analysis, or for drainage of the fluids in the case of peripheral oedemas.

The present invention also recognises a use to disrupt epithelial barrier electrical resistance for the improvement of signal to noise ratio in electrophysiological recordings. The epithelial barrier electrical resistance may be recorded through the skin or other epithelial surface, for example during an electrocardiogram, electro-encephalogram, electro-retinogram, or electromyogram.

More detail regarding the molecules that can be delivered and the technologies that can benefit from this invention are detailed below.

### DETAILED DESCRIPTION OF THE INVENTION

### The Method

The method may comprise the application of the payload molecule to the epithelial tissue barrier together with the agent capable of disrupting the epithelial tissue barrier. In another embodiment, the method may comprise the step of disrupting the epithelial tissue barrier function by contact with the agent capable of disrupting the epithelial tissue barrier, and then applying the payload molecule to the disrupted epithelial tissue barrier.

In general, the payload molecule may be applied before, concurrently with, or subsequent to the application of the agent capable of disrupting the epithelial tissue barrier. The application of the payload molecule and the agent may be simultaneous, sequential or separate.

Disrupting the epithelial tissue barrier function may comprise or consist of reducing the trans-epithelial electrical resistance (TEER) of the epithelial tissue barrier. In one embodiment, disrupting the epithelial tissue barrier function comprises or consists of the diminution of diffusive resistance/restriction across the epithelial barrier. In another embodiment, disrupting the epithelial tissue barrier function comprises or consists of reducing the electrical resistance (TEER) and/or hydraulic resistance of the epithelial tissue barrier.

The method of the invention may be carried out in vivo, for example in a subject, or in vitro, for example in a tissue model or extract. The method of the invention may be a treatment of a subject for a disorder, or a preventative therapy.

The method may involve topical delivery to directly deliver the payload molecule to a local site, for example there may be topical application to the eye to deliver a drug for treating an eye condition.

It may alternatively be that the method may involve topical delivery to deliver the payload molecule into the body of the subject for onward delivery to a site that is distinct from the application site. Thus, there may be systemic distribution of the payload molecule to a downstream location, e.g. by delivery of the payload molecule to the subject's bloodstream.

One example is the administration of a payload molecule to the skin adjacent to the nail of a subject, such that the payload molecule may then enter the blood supply extending below the nail, e.g. to target a fungal infection.

In general, systemic or compartmental biodistribution could be targeted by a focal skin (or other barrier) administration, exploiting the subject's circulation to deliver the payload molecule downstream or systemically.

### Agents Capable of Disrupting the Epithelial Tissue Barrier

In general, the agent capable of disrupting the epithelial tissue barrier may be a carboxylic acid compound of Formula (I), or a pharmaceutically acceptable salt, hydrate or solvate thereof, which includes at least one R′ group, or it may be a microbial quorum sensing signalling molecule (QSSM) or a derivative or variant thereof which is a compound that comprises a heterocyclic ring portion and an alkane portion comprising an R′ group. In each case the R′ group is a C1-12 alkyl group, that may be unsubstituted or substituted, wherein any substituent groups that are present are independently selected from hydroxyl, halogen and NR″₂, where each R″ is independently selected from hydrogen and methyl.

The microbial QSSM may be a bacterial QSSM. A derivative or variant of a microbial QSSM may be synthetic (i.e. non-natural). In particular, the derivative or variant of a microbial QSSM may be synthetically/artificially produced. The derivative or variant of a microbial QSSM may or may not be produced or extracted from a microbe.

In one embodiment, the agent capable of disrupting the epithelial tissue barrier is the P. aeruginosa quorum-sensing molecule N-(3-oxododecanoyl) homoserine lactone (30C12-HSL).

In another embodiment, the agent capable of disrupting the epithelial tissue barrier is the 2-N-heptyl-3-hydroxy-4 (1H)-quinolone (C7 PQS).

In another embodiment, the agent capable of disrupting the epithelial tissue barrier is any one of the agents shown in Table 1 in the Examples as being effective in enhancing translocation of 4 kDa FITC-dextran (i.e. indicated as “Yes” in the final column of the table).

In another embodiment, the agent capable of disrupting the epithelial tissue barrier is any one of the agents shown in Table 2 in the Examples as being an agent that causes MMP activation.

The agent capable of disrupting the epithelial tissue barrier, such as the microbial QSSM, may be provided as a solvate in solution.

The agent capable of disrupting the epithelial tissue barrier, such as the microbial QSSM, may or may not be encapsulated or complexed within a carrier particle, such as a nanostructured lipid particle (NLP).

The agent capable of disrupting the epithelial tissue barrier, such as the microbial QSSM, may, in one embodiment, be encapsulated (trapped) or impregnated within a polymer, or adsorbed to a particle, macromolecular carrier or protein.

The agent capable of disrupting the epithelial tissue barrier, such as the microbial QSSM, is crystallised, lyophilised or otherwise manufactured into nano-objects (i.e. particles in the 1 nm to 1000 nm range), such as lipid micelles, droplets, or vesicles. In another embodiment, the agent capable of disrupting the epithelial tissue barrier, such as the microbial QSSM, is bound or adsorbed onto the surface of nano-objects (i.e. particles in the 1 nm to 1000 nm range), such as lipid micelles, droplets, or vesicles.

In one embodiment, the agent may be used in an amount of 1 μM or more, such as 5 μM or more, or 10 μM or more, or 50 μM or more; e.g. 100 μM or more, or 200 μM or more.

In one embodiment, the agent may be used in an amount of 1 to 5000 μM, such as 5 to 3000 μM, or 10 to 200 μM, or 50 to 1000 μM; e.g. 100 to 750 μM, or 200 to 500 μM.

### R′ Group

R′ is present in each of the series of agents according to the invention and each of Formulae (I)-(V) and is independently a C1-12 alkyl group, that may be unsubstituted or substituted.

It will be appreciated that each R′ does not include any double bonds, because double bonds are not present in an alkyl group, nor are they present in any of the permitted substituent groups. The R′ group is saturated. The R′ group is therefore flexible.

A straight alkyl chain which is flexible has been found to be essential for agents to have effective activity in disrupting the epithelial tissue barrier. Conformational restrictions introduced through double bonds (cis or trans) are significantly detrimental to activity.

The R′ group is preferably a saturated hydrocarbon chain with no N heteroatoms included within the chain. In particular, it has been identified that the conformational restrictions introduced through amide functionality (meaning there is an N within the hydrocarbon chain), are detrimental and adversely reduce the activity.

In one embodiment, the R′ group is preferably a saturated hydrocarbon chain with no N or S heteroatoms included within the chain. In one embodiment, the R′ group is preferably a saturated hydrocarbon chain with no N or O heteroatoms included within the chain. In one particular embodiment, the R′ group is a saturated hydrocarbon chain with no N or S or O heteroatoms included within the chain. Thus, it may be that the backbone of the R′ group chain is based only on carbon atoms.

The hydrocarbon chain may optionally have substituent groups but any substituent groups that are present must be independently selected from hydroxyl, halogen and NR″₂, where each R″ is independently selected from hydrogen and methyl. Thus sterically bulky substituents are not permitted and the substituents that are present do not have any significant adverse effect on the flexibility of the chain. In one embodiment there are from 1 to 4 substituent groups, such as 1 or 2 substituent groups.

In one embodiment, any substituent groups that are present are independently selected from hydroxyl and halogen.

In one preferred embodiment, any substituent groups that are present are independently selected from hydroxyl and F. It may be that the only substituent groups that are present are —OH. It may be that the only substituent groups that are present are F.

In one preferred embodiment, the C1-12 alkyl group is unsubstituted.

An unsubstituted or minimally substituted C1-12 alkyl group is flexible and this therefore contributes to having excellent activity in disrupting the epithelial tissue barrier.

When R′ is a C3-12 alkyl group, it may optionally be branched but preferably it has no more than a single C1 branch off the main chain (i.e. the main chain comprises all, or all bar one, of the carbon atoms) and more preferably the alkyl group is unbranched. Thus, in one embodiment, each R′ is independently an unbranched C1-12 alkyl group. In another embodiment, each R′ is independently a branched C3-12 alkyl group where the branch is a C1 alkyl (methyl) group.

The conformational restrictions introduced through branching are detrimental and adversely reduce the activity. Thus only C1 branching can be tolerated; greater degrees of branching lead to a noticeable reduction in activity. It may be preferred that there is no branching, in order to achieve optimal activity.

It will be understood that R′ may be C1, C2, C3, C4, C5, C6, C7, C8, C9, C10, C11 or C12 alkyl, and is optionally substituted but preferably unsubstituted. In one embodiment, R′ is C1-11 alkyl, and is optionally substituted but preferably unsubstituted.

In one embodiment, R′ may be selected from C1 and C6-C12 alkyl, and is optionally substituted but preferably unsubstituted. In one embodiment, R′ may be selected from C1 and C7-C12 alkyl, and is optionally substituted but preferably unsubstituted. In one embodiment, R′ may be selected from C1, C7, C9 and C11, and is optionally substituted but preferably unsubstituted.

In one embodiment, R′ may be unsubstituted C₁-C₁₂ alkyl, or unsubstituted C₁-C₁₁ alkyl, e.g. unsubstituted C6-C12 alkyl or unsubstituted C7-C11 alkyl.

R′ may in one embodiment be an unsubstituted methyl group; or a methyl group which is substituted with one substituent group; or may be an unsubstituted ethyl group.

If the R′ group is provided as a substituent group on an aromatic or non-aromatic ring, then it is preferred that there is not a CH₂NMe substituent at any position that is ortho to the R′ group.

In one embodiment, the substituents at any position ortho to the R′ group are selected from the group consisting of: OH, CH₃, H, R′, and O.

In one embodiment, the R′ group is provided as a substituent group on an aromatic or non-aromatic heterocylic ring that includes a N in the ring. It may be that the N is at a location ortho to the R′ substituent group. It may be that the N is substituted with H, R′, or O. It may be that the other position ortho to the R′ substituent group is a C in the ring that may be substituted with OH, CH₃ or H.

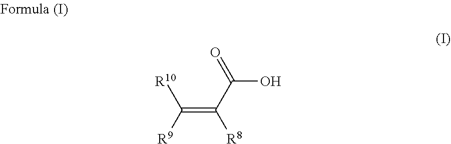

Formula (I) is a carboxylic acid compound. It can be characterised as being an alkene having a carboxylic acid substituent and having least one R′ group substituent.

In this regard, R⁸ is H or R′; R⁹ is H or R′; and R¹⁰ is H or R′, provided that at least one R′ group is present. In one preferred embodiment, only one R′ group is present.

Formula (I) therefore has an alkane “tail” portion comprising an R′ group which flexible. It also has a carboxylic acid plus alkene portion; this therefore provides a less flexible/more rigid portion which includes at least one heteroatom.

The alkene may have E or Z stereochemistry. In some embodiments the alkene may have Z stereochemistry, i.e. a cis double bond. This is typical of an unsaturated fatty acid.

It is preferred that no other double bonds are present. In particular, the definition of the R′ group substituent for the present invention does not permit the presence of double bonds. In this regard, the R′ group is a C1-12 alkyl group, that may be unsubstituted or substituted, wherein any substituent groups that are present are independently selected from hydroxyl, halogen and NR″₂, where each R″ is independently selected from hydrogen and methyl.

As noted above, a straight alkyl chain which is flexible has been found to be essential for agents to have effective activity in disrupting the epithelial tissue barrier. Conformational restrictions introduced through double bonds (cis or trans) other than those shown in Formula (I) are significantly detrimental to activity.

In some embodiments at least one of R⁸ and R⁹ is H. In some preferred embodiments R⁸ is H and R⁹ is H (and therefore R¹⁰ is R′).

It may be that the compound is cis-2-decenoic acid. In other embodiments, the compound is not cis-2-decenoic acid.

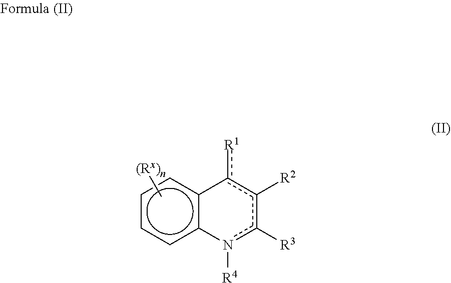

Formula (II) comprises two six-membered rings that share two adjacent carbon atoms (known as bridging atoms), i.e. a fused bicyclic structure.

The first ring is an aromatic or alicyclic carbon ring that is optionally substituted.

In this regard, each substituent group R^{x} for the first ring is independently selected from halo, C1-12 alkyl, C1-12 alkylhalo, OR^{y}, and NR″₂, and where R^{y} is hydrogen or C1-12 alkyl, and where each R″ is independently selected from hydrogen and methyl.

The first ring may be substituted (n=1-4) or unsubstituted (n=0).

In one embodiment, when the first ring is substituted it may have one or more substituents selected from C1-12 alkyl, C1-12 alkoxy, hydroxyl, and halogen. In one embodiment, when the first ring is substituted it may have one or more substituents selected from C1-6 alkyl, C1-6 alkoxy, hydroxyl, and halogen (e.g. F).

In one preferred embodiment, when the first ring is substituted it may have one or more substituents selected from hydroxyl and halogen (e.g. F), e.g. there may be one or two substituents selected from hydroxyl and halogen (e.g. F).

In one preferred embodiment there is a halogen (e.g. F) substitution in position 7; this has been found to enhance the activity of the agent. In particular, this is the case when there is an OH group at the R² position. Without being bound by theory, this may be due to increasing the polarizability of the OH at the R² position.

There are effective results achieved with a halogen (e.g. F) substitution in position 6. However, in one embodiment when there is an OH group at the R² position there is no halogen (e.g. F) substitution in position 6. Without being bound by theory, it is believed that this may cause some reduction in the polarizability of the OH at the R² position.

In one embodiment, when there is an OH group at the R² position on the second ring there is one or more substituent on the first ring; for example there may be one or more substituents selected from hydroxyl and halogen (e.g. F) on the first ring.

The first ring may, in one embodiment, be aromatic, as shown in Formula (IIA) below.

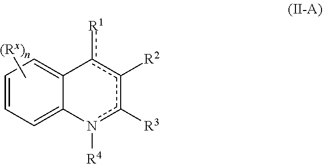

In Formula (II), the second ring comprises five carbon atoms and one nitrogen atom, the nitrogen atom being located adjacent to a bridging atom.

The second ring is substituted. In this regard, R¹ is O or OH; R² is OH, CH₃ or H; R³ is R′; R⁴ is H, R′, or O.

The second ring is therefore substituted with at least one R′ group; in a preferred embodiment it is substituted with only one R′ group (i.e. R⁴ is H or O).

Formula (II) therefore has an alkane “tail” portion comprising an R′ group which flexible. It also has a heterocyclic ring portion; this therefore provides a less flexible/more rigid portion which includes at least one heteroatom.

In Formula (II), the or each R′ group is most preferably a C7 group in terms of optimal activity; however other chain lengths are also effective. Thus, for example, the or each R′ group may be a C1-11 alkyl group, such as a C1-7 alkyl group, which is optionally substituted but preferably unsubstituted.

The small substituent group (OH, CH₃ or H) at the R² position, i.e. adjacent to the R′ group, is believed to be important in the structure-activity relationship. In this regard, it has been shown that the presence of a basic functional group, such as a F, —NH₂ or—CH₂NMe substituent, at the R² position is highly detrimental to the efficacy of the agent, considerably reducing the activity of the agent. The acidic substituent group OH at the R² position is associated with excellent activity.

The second ring comprises at least one double bond. The skilled person will appreciate that the location of the double bond(s) may depend on the substituents R¹ to R⁴.

For example, the second ring could have one double bond (as in Formulae (II-B) and (II-C)) or could have two double bonds (as in Formulae (II-D) and (II-E)):

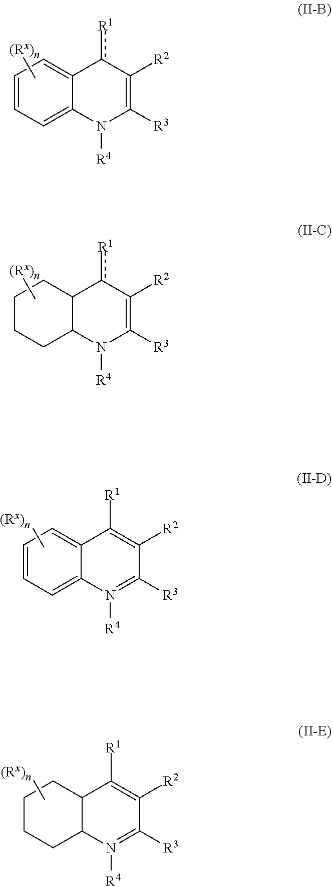

In some embodiments, R¹ may be O, such that the second ring comprises a carbonyl group (C═O) adjacent a bridging atom (as in Formula (II-B) and Formula (II-C)). In some embodiments where R¹ is O, R² may be OH or H and/or R⁴ may be H or R′.

Compounds where R¹ is O, R² is OH and R⁴ is H may usefully be chosen for use as the agent; these include C7 PQS, C1 PQS, C9 PQS, and C11 PQS, which are shown below.

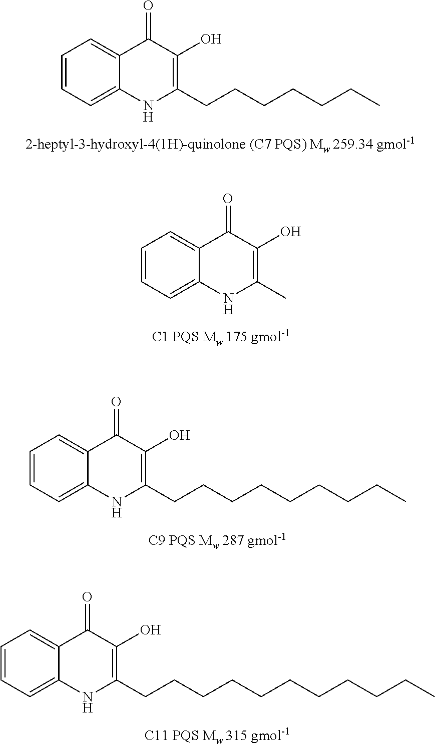 [SMILES: CCCCCCCc1[nH]c2ccccc2c(=O)c1O ; Cc1[nH]c2ccccc2c(=O)c1O ; CCCCCCCCCc1[nH]c2ccccc2c(=O)c1O ; CCCCCCCCCCCc1[nH]c2ccccc2c(=O)c1O]

Compounds where R¹ is O, R² is H, and R⁴ is H may usefully be chosen for use as the agent; these include HHQ, NHQ, and UHQ, which are shown below.

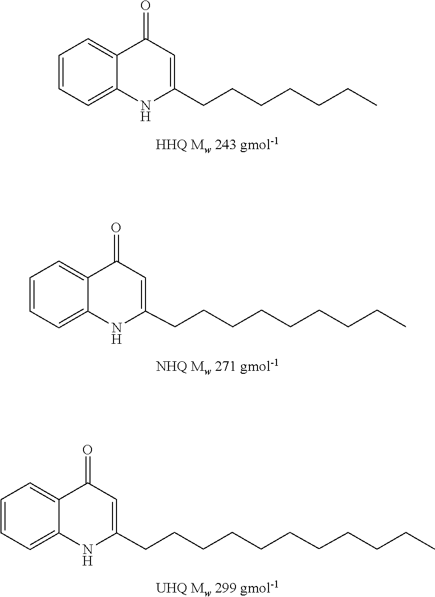 [SMILES: CCCCCCCc1cc(=O)c2ccccc2[nH]1 ; CCCCCCCCCc1cc(=O)c2ccccc2[nH]1 ; CCCCCCCCCCCc1cc(=O)c2ccccc2[nH]1]

In one embodiment, R¹ may be OH (hydroxyl). In some embodiments where R¹ is OH, R² may be H and/or R⁴ may be O. For example, the compound may have a structure as in Formula (II-D) or Formula (II-E), as shown above.

In some embodiments, R⁴ may be O, to provide an N-oxide (also known as an amine oxide). An N-oxide comprises N⁺-O⁻, which can be represented with an arrow, as in Formula (II-F). An aromatic first ring is illustrated, but the first ring could be alicyclic.

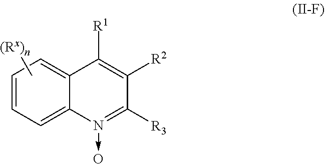

In particular, R¹ may be OH and R⁴ may be O to provide a compound having a structure as shown in Formula II-G. An aromatic first ring is illustrated, but the first ring could be alicyclic.

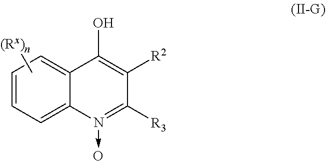

In some embodiments where R⁴ is O, R¹ may be OH and/or R² may be H or R′. Compounds where R⁴ is O, R¹ is OH and R² is H may usefully be chosen for use as the agent; these include C7 HHQ N-oxide and HNQ N-oxide, which are shown below.

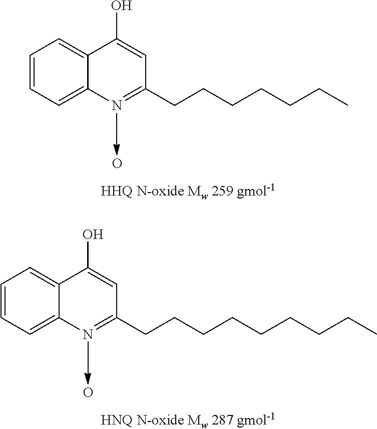 [SMILES: CCCCCCCc1cc(O)c2ccccc2n1 ; O ; CCCCCCCCCc1cc(O)c2ccccc2n1 ; O]

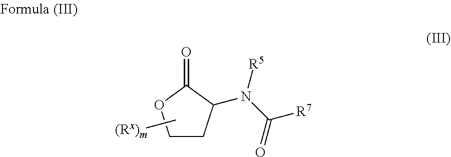

Formula (III) comprises a lactone moiety, i.e. a cyclic carboxylic ester.

The heterocyclic ring is optionally substituted. In this regard, each substituent group R^{x} for the ring is independently selected from halo, C1-12 alkyl, C1-12 alkylhalo, OR′, and NR″₂, and where R^{y} is hydrogen or C1-12 alkyl, and where each R″ is independently selected from hydrogen and methyl.

The ring may be substituted (n=1-4) or unsubstituted (n=0).

In one embodiment, when the ring is substituted it may have one or more substituents selected from C1-12 alkyl, C1-12 alkoxy, hydroxyl, and halogen. In one embodiment, when the ring is substituted it may have one or more substituents selected from C1-6 alkyl, C1-6 alkoxy, hydroxyl, and halogen (e.g. F). In one embodiment, when the ring is substituted it may have one or more substituents selected from hydroxyl and halogen (e.g. F).

When considering the remainder of the molecule, R⁵ is H or R′; R⁶ is O, CH₂, CHR′ or CR′2; and R⁷ is CH₂C(O) R′ or R′. Thus, due to the R⁷ group, there is at least one R′ group present; in a preferred embodiment there is only one R′ group present in the molecule.

Formula (III) therefore has an alkane “tail” portion comprising an R′ group which flexible. It also has a heterocyclic ring portion; this therefore provides a less flexible/more rigid portion which includes at least one heteroatom.

In one embodiment, R⁶ may be CH₂, CHR′ or CR′2 to provide an alkene, which may have Z or E stereochemistry. In some embodiments where R⁶ is selected from CH₂, CHR′ or CR′2, R⁵ may be H and/or R⁷ may be R′.

In one embodiment, R⁶ may be O to provide a carbonyl group (C═O). Compounds where R⁵ is H, R⁶ is O and R⁷ is R′ may usefully be chosen for use as the agent; these include C4 HSL, also known as N-butyl-homoserine-lactone (BHL):

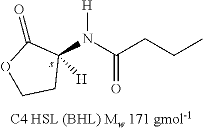

In some embodiments where R⁶ is O, R⁵ may be H and/or R⁷ may be CH₂C(O) R′. Compounds where R⁵ is H, R⁶ is O, and R⁷ is CH₂C(O) R′ may usefully be chosen for use as the agent; these include HSL and 3-oxo-C12-HSL:

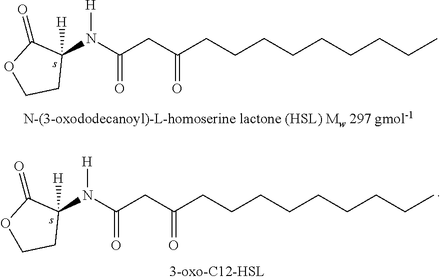 [SMILES: CCCCCCCCCC(=O)CC(=O)N[C@H]1CCOC1=O ; CCCCCCCCCC(=O)CC(=O)N[C@H]1CCOC1=O]

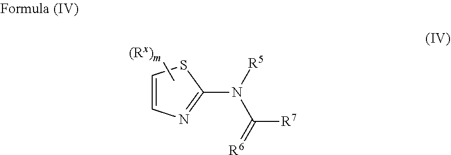

Formula (IV) includes a thiazole ring. The thiazole ring is optionally substituted. In this regard, each substituent group R^{x} for the ring is independently selected from halo, C1-12 alkyl, C1-12 alkylhalo, OR^{y}, and NR″₂, and where R^{y} is hydrogen or C1-12 alkyl, and where each R″ is independently selected from hydrogen and methyl.

The ring may be substituted (m=1 or 2) or unsubstituted (m=0).

In one embodiment, when the ring is substituted it may have one or more substituents selected from C1-12 alkyl, C1-12 alkoxy, hydroxyl, and halogen. In one embodiment, when the ring is substituted it may have one or more substituents selected from C1-6 alkyl, C1-6 alkoxy, hydroxyl, and halogen (e.g. F). In one embodiment, when the ring is substituted it may have one or more substituents selected from hydroxyl and halogen (e.g. F).

When considering the remainder of the molecule, R⁵ is H or R′; R⁶ is O, CH₂, CHR′ or CR′2; and R⁷ is CH₂C(O) R′ or R′. Thus, due to the R⁷ group, there is at least one R′ group present; in a preferred embodiment there is only one R′ group present in the molecule.

Formula (IV) therefore has an alkane “tail” portion comprising an R′ group which flexible. It also has a heterocyclic ring portion; this therefore provides a less flexible/more rigid portion which includes at least one heteroatom.

In one embodiment, R⁶ may be CH₂, CHR′ or CR′2 to provide an alkene, which may have Z or E stereochemistry. In some embodiments where R⁶ is selected from CH₂, CHR′ or CR′2, R⁵ may be H and/or R⁷ may be R′.

In one embodiment, R⁶ may be O to provide a carbonyl group (C═O). Compounds where R⁵ is H, R⁶ is O and R⁷ is R′ may usefully be chosen for use as the agent.

In some embodiments where R⁶ is O, R⁵ may be H and/or R⁷ may be CH₂C(O) R′. Compounds where R⁵ is H, R⁶ is O, and R⁷ is CH₂C(O) R′ may usefully be chosen for use as the agent.

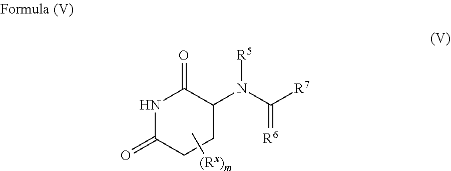

Formula (V) includes a piperidine ring. The ring is optionally substituted. In this regard, each substituent group R^{x} for the ring is independently selected from halo, C1-12 alkyl, C1-12 alkylhalo, OR^{y}, and NR″₂, and where R^{y} is hydrogen or C1-12 alkyl, and where each R″ is independently selected from hydrogen and methyl.

The ring may be substituted (m=1 or 2) or unsubstituted (m=0).

In one embodiment, when the ring is substituted it may have one or more substituents selected from C1-12 alkyl, C1-12 alkoxy, hydroxyl, and halogen. In one embodiment, when the ring is substituted it may have one or more substituents selected from C1-6 alkyl, C1-6 alkoxy, hydroxyl, and halogen (e.g. F). In one embodiment, when the ring is substituted it may have one or more substituents selected from hydroxyl and halogen (e.g. F).

When considering the remainder of the molecule, R⁵ is H or R′; R⁶ is O, CH₂, CHR′ or CR′2; and R⁷ is CH₂C(O) R′ or R′. Thus, due to the R⁷ group, there is at least one R′ group present; in a preferred embodiment there is only one R′ group present in the molecule.

Formula (V) therefore has an alkane “tail” portion comprising an R′ group which flexible. It also has a heterocyclic ring portion; this therefore provides a less flexible/more rigid portion which includes at least one heteroatom.

In one embodiment, R⁶ may be CH₂, CHR′ or CR′2 to provide an alkene, which may have Z or E stereochemistry. In some embodiments where R⁶ is selected from CH₂, CHR′ or CR′2, R⁵ may be H and/or R⁷ may be R′.

In one embodiment, R⁶ may be O to provide a carbonyl group (C═O). Compounds where R⁵ is H, R⁶ is O and R⁷ is R′ may usefully be chosen for use as the agent.

In some embodiments where R⁶ is O, R⁵ may be H and/or R⁷ may be CH₂C(O) R′. Compounds where R⁵ is H, R⁶ is O, and R⁷ is CH₂C(O) R′ may usefully be chosen for use as the agent.

### The Payload Molecule

In one embodiment, the payload molecule is a therapeutic agent, such as a macromolecular therapeutic agent. It is unexpected that a range of sizes of payload molecule can be effectively delivered using the present invention rather than only small molecules such as dextrin (4 kDa M_{w}). Molecules that have a M_{w} of, for example, 10 kDa or more, and as high as 150 kDa or more, can effectively be delivered by penetrating the epithelial barrier in the presence of the agent according to the invention. The payload molecule may not naturally be able to penetrate the epithelial barrier in the absence of the agent according to the invention.

The payload molecule may comprise a protein. The payload molecule may comprise a peptide or protein. The payload molecule may comprise a physiologically or metabolically relevant protein or peptide. The protein may be a glycoprotein. The protein or glycoprotein may be an enzyme. In another embodiment, the payload molecule may comprise a polysaccharide. In another embodiment, the payload molecule may comprise botox.

The payload molecule may comprise a non-small molecule (e.g. M_{w}>900 Da) or a small molecule (e.g. M_{w}<900 Da). The payload molecule may be any molecule of M_{w} less than 200 kDa. In another embodiment, the payload molecule may be any molecule of M_{w} less than 150 kDa. In another embodiment, the payload may have a M_{w} between 1 kDa and 150 kDa.

The payload molecule may comprise a signalling protein, which is a protein involved in a signal pathway. The payload molecule may comprise a protein involved with regulation of expression or metabolism of a cell. The payload molecule may comprise a protein involved with cell division. The payload molecule may comprise a marker, such as a protein marker. The payload molecule may comprise a bacterial, or bacterially-derived protein. The payload molecule may comprise a mammalian, or mammalian-derived protein. The payload molecule may be any peptide, polypeptide or protein. The payload molecule may comprise research, diagnostic or therapeutic molecules. The payload molecule may comprise an enzyme or substrate thereof, a protease, an enzyme activity modulator, a perturbimer and peptide aptamer, an antibody, a modulator of protein-protein interaction, a growth factor, or a differentiation factor. The payload molecule may be a pre-protein or pro-drug. The payload molecule may comprise a viral particle or virus-like particle. In one embodiment, the payload molecule is an antibody, or antibody fragment or mimetic, such as a nanobody.

In one embodiment, the payload molecule is selected from any of the group comprising a therapeutic molecule; a drug; a pro-drug; a functional protein or peptide, such as an enzyme or a transcription factor; a microbial protein or peptide; a viral particle; a virus-like-particle; and a toxin; or nucleic acid encoding thereof.

In one embodiment, the payload molecule may comprise nucleic acid, such as siRNA, messenger RNAs (mRNAs), micro RNAs or DNA constructs. The payload molecule may comprise nucleic acid complex. The nucleic acid may be recombinant.

The payload molecule may comprise non-covalently bound complexes such as protein-protein complexes, protein-mRNA, protein-non-coding RNA, protein-lipid and protein-small molecule complexes.

The payload molecule may be a chemotherapeutic. In one embodiment, the payload molecule is an anti-VEGF antibody, or fragment thereof, or an inhibitor of VEGF. In one embodiment, the payload molecule is selected from the group consisting of bevacizumab (Avastin®), aflibercept (Eylea®) and doxorubicin. These are non-limiting examples, and the skilled person will appreciate that the present invention can be applied to a range of payload molecules with a range of molecular weights.

In another embodiment, the payload molecule is an anti-inflammatory. In one embodiment, the payload molecule comprises a cytokine, such as interferon beta, preferably interferon beta 1a (20 kDa). The cytokine, such as interferon beta, may be a cytokine that is about 25 kDa or less. The skilled person will recognise that interferon beta may be used to treat or prevent inflammation, such as inflammation in asthma disorders, or the effects of viral infection, such as covid-19 infection.

In one embodiment, the payload molecule is a therapeutic agent that is suitable for treatment or prevention of a disorder in an organ or tissue having an epithelial tissue barrier.

In one embodiment, the payload molecule is a therapeutic agent that is suitable for treatment or prevention of an eye disorder. The eye disorder may comprise any one of the disorders selected from macular degeneration, cancer, diabetic retinopathy, retinal vascular occlusive disease, inflammatory eye disease, and retinopathy of prematurity. In one embodiment, the eye disorder comprises macular degeneration, such as AMD (age-related macular degeneration). The AMD may be wet-AMD or dry-AMD. In another embodiment, the eye disorder comprises cancer, such as a solid tumour cancer, melanoma, and retinoblastoma. In another embodiment, the eye disorder comprises diabetic retinopathy. In another embodiment, the eye disorder comprises retinal vascular occlusive disease, such as venous stasis retinopathy, central retinal vein occlusion, and branch retinal vein occlusion. In another embodiment, the eye disorder comprises inflammatory eye disease, such as iritis, uveitis, scleritis, blepharitis and orbital inflammatory disease. In another embodiment, the eye disorder comprises retinopathy of prematurity.

In another embodiment, the payload molecule is a therapeutic agent that is suitable for treatment or prevention of a respiratory disorder, such as mepolizumab. The respiratory disorder may comprise any one of the disorders selected from microbial infection, cancer, asthma, interstitial lung disease, inflammatory lung disease, tumours and COPD. In one embodiment, the respiratory disorder comprises microbial infection, or associated inflammatory effects of microbial infection. In one embodiment, the microbial infection is a viral infection, such as coronavirus infection. The coronavirus may comprise SARS-COV-2 (Covid-19). The viral infection may comprise influenza. In another embodiment, the microbial infection is a bacterial infection. The bacterial infection may be Bacillus anthracis infection. In another embodiment, the microbial infection is a fungal infection. The fungal infection may comprise aspergillus infection.

The respiratory disorder may comprise inflammatory disease, such as asthma. The respiratory disorder may comprise COPD (chronic obstructive pulmonary disease).

In another embodiment, the respiratory disorder comprises cancer, for example lung cancer.

In another embodiment, the payload molecule is a therapeutic agent that is suitable for treatment or prevention of a gastrointestinal disorder. The gastrointestinal disorder may comprise any one of the disorders selected from inflammatory bowel disease, cancer, such as bowel cancer, oesophageal cancer, mouth cancer, tongue cancer, or stomach cancer. In one embodiment, the gastrointestinal disorder comprises inflammatory bowel disease, such as Crohn's disease and ulcerative colitis. The therapeutic agent suitable for the treatment of inflammatory bowel disease may comprise aminosalicylates (such as sulphasalazine, mesalazine, olsalazine and balsalazide), steroids (such as prednisolone, prednisone, hydrocortisone, methylprednisolone, beclometaone dipropionate, budesonide, budesonide-MMX), immunosuppressants (such as ciclosporin, azathioprine, mercaptopurine, and methotrexate), biological or biosimilar medicines (such as adalimumab, golimumab, vedolizumab, ustekinumab, and infliximab), or small molecules (such as tofacitinib).

In one embodiment, the payload molecule is a therapeutic agent that is suitable for treatment or prevention of a skin disorder. The skin disorder may comprise any one of the disorders selected from microbial infection, such as bacterial or fungal skin infections, skin neoplasms and inflammatory skin disorders. In one embodiment the skin disorder comprises a skin neoplasm, which may be cancerous. The cancerous skin neoplasm may be a cancer, such as skin cancer (e.g. melanoma), or solid tumours presenting in the dermis or subcutaneous tissue. In another embodiment, the skin disorder comprises inflammatory skin disorders, such as eczema, acne and psoriasis.

The therapeutic agent suitable for the treatment of the skin disorder may comprise small molecules (such as lignocaine and triamcinolone), biologics (such as adalimumab) or plasmid gene therapy.

In one embodiment, the payload molecule may be a prophylactic agent, such as a vaccine.

The payload molecule, such as a therapeutic agent, may be provided at a therapeutically or prophylactically effective concentration.

Combinations of payload molecules may be provided. The payload molecule, such as a therapeutic agent, may be provided in combination with one or more other therapeutically active agents. For example, a second, third or more therapeutic agent may be provided.

In one embodiment, the payload molecule may have a molecular weight (M_{w}) of at least 0.3 kDa, e.g. 0.4 kDa or more. In one embodiment, the payload molecule may have a molecular weight of at least 0.5 kDa. In another embodiment, the payload molecule may have a molecular weight of at least 1 kDa. The payload molecule may have a molecular weight of at least 4 kDa. Alternatively, the payload molecule may have a molecular weight of at least 5 kDa.

In one embodiment, the payload molecule may have a molecular weight (M_{w}) of at least 10 kDa. In one embodiment, the payload molecule may have a molecular weight of at least 15 kDa. In another embodiment, the payload molecule may have a molecular weight of at least 20 kDa.

In one embodiment, the payload molecule may have a molecular weight (M_{w}) of at least 25, or at least 50, or at least 100 kDa.

The payload molecule may, in some embodiments, have a molecular weight (M_{w}) of 400 kDa or less. The payload molecule may have a molecular weight of 300 kDa or less.

In one non-limiting example the payload molecule may have a molecular weight (M_{w}) of between about 0.5 kDa and about 400 kDa; or between about 1 kDa and about 300 kDa; or between about 1 kDa and about 250 kDa; or between about 4 kDa and about 250 kDa; or between about 5 kDa and about 200 kDa; or between about 10 kDa and about 200 kDa. The payload molecule may have a molecular weight (M_{w}) of between about 50 kDa and about 200 kDa; or between about 100 kDa and about 150 kDa.

Where the payload molecule comprises amino acids, the payload molecule may be between about 5 and about 30,000 amino acids in length, or more. The payload molecule may be between about 5 and about 10,000 amino acids in length. The payload molecule may be between about 5 and about 5,000 amino acids in length. The payload molecule may be between about 5 and about 1000 amino acids in length. The payload molecule may be at least about 5 amino acids in length. The payload molecule may be at least about 100 amino acids in length.

The skilled person will recognise that the ability of a payload molecule to cross an epithelial barrier can be dependent on its size, shape and/or charge.

The invention may be suitable for enhancing transport of any payload molecule. For example, the invention may be particularly beneficial in relation to those molecules where the spontaneous rate of crossing the epithelial barrier is too low to be therapeutically effective without enhancement of the delivery.

### The Epithelial Tissue Barrier

The epithelial tissue barrier may be in vivo or in vitro. The epithelial tissue barrier may be in situ in a subject.

The epithelial tissue barrier may be in the brain, eye, respiratory system, skin, gastrointestinal tract, urinary tract, male or female reproductive system, olfactory system, or any mucous membrane of a subject. In one embodiment, the epithelial tissue barrier is in the eye, for example, the epithelial tissue barrier may be corneal and conjunctival epithelium. In another embodiment, the epithelial tissue barrier may be an internal barrier, such as the blood-brain barrier, blood-testicular barrier, or blood-retinal barrier.

In another embodiment, the epithelial tissue barrier is in the respiratory system, for example, the epithelial tissue barrier may comprise lung epithelia.

In another embodiment, the epithelial tissue barrier is in the gastrointestinal tract, for example, the epithelial tissue barrier may comprise epithelia of the mouth (buccal epithelia), oesophagus, stomach, intestine (small and/or large), or rectum.

In another embodiment, the epithelial tissue barrier is in the urinary tract, for example, the epithelial tissue barrier may comprise epithelia of the urethra or bladder. In one embodiment, the epithelial tissue barrier is in the kidney.

In another embodiment, the epithelial tissue barrier is in the female reproductive tract, for example, the epithelial tissue barrier may comprise epithelia of the ovaries, fallopian tubes, uterus, or cervix.

In another embodiment, the epithelial tissue barrier may comprise olfactory/nasal epithelium.

In another embodiment, the epithelial tissue barrier may comprise the epithelium of the tympanic membrane.

### The Subject

The subject may be mammalian. In one embodiment, the subject is a human subject. The subject may be male or female. The subject may be a non-human animal, such as a domestic animal or livestock. In one embodiment, the use of the invention may be veterinary, or in animal research. The subject may be a rodent, rabbit or simian.

In one embodiment, the subject may be in need of treatment for an eye disorder or may be at risk of developing an eye disorder. In another embodiment the subject may be in need of treatment for a respiratory disorder or may be at risk of developing a respiratory disorder. In another embodiment the subject may be in need of treatment for a gastro-intestinal disorder or may be at risk of developing a gastro-intestinal disorder. In another embodiment the subject may be in need of treatment for a urinary tract disorder or may be at risk of developing a urinary tract disorder. In another embodiment the subject may be in need of treatment for a disorder of the reproductive system or may be at risk of developing a disorder of the reproductive system. In another embodiment the subject may be in need of treatment for a skin disorder or may be at risk of developing a skin disorder. In another embodiment the subject may be in need of treatment for a brain disorder or may be at risk of developing a brain disorder. In another embodiment the subject may be in need of treatment for an auto-immune disorder.

It will be appreciated that the invention may also be used to achieve systemic concentrations of a payload molecule in order to treat a systemic disease. The invention may also be used to deliver a payload molecule into specific compartments or tissues of the body, such as via epithelial barriers that may separate such compartments or structures of the body. For example, a payload molecule may be delivered to the brain via the nose.

The subject may be in need of a more comfortable delivery system than invasive delivery methods, such as injections. For example, the subject may benefit from a payload molecule delivery via a topically applied patch or a topical composition such as a gel.

### Compositions

The composition according to the invention may comprise a carrier. The skilled person will appreciate that the carrier may suitably be pharmaceutically acceptable.

In one embodiment, the composition is a pharmaceutically acceptable composition that is suitable for systemic delivery, such as intravenous delivery.

In another embodiment, the composition is a pharmaceutically acceptable composition that is suitable for topical delivery, such as application to the skin or to the surface of the eye.

The composition may be an ophthalmically acceptable composition. Any carrier present may therefore be an ophthalmically acceptable carrier. The composition may be suitable for topical administration to the eye.

In one embodiment, the composition is an ophthalmic composition. An ophthalmic composition is understood to be a sterile, liquid, semi-solid, or solid preparation that may contain one or more active pharmaceutical ingredient(s) intended for application to the eye or eyelid.

The composition may be in the form of the agent capable of disrupting the epithelial tissue barrier suspended in a gel, lotion, cream, ointment, or solution, such as an aqueous solution. In one embodiment, the composition is in the form of drops, such as an eye drop formulation. In one embodiment, the composition is formulated with a gel, such as a pluronic/poloxamer gel. The pluronic/poloxamer gel may comprise poloxamer 407 (F127). The composition may be in the form of a spray. The composition may be formulated for oral delivery, for example for sublingual delivery or in a cheek patch. The composition may be formulated for ocular delivery. In one embodiment, the composition may be provided in a contact lens, for example for ocular delivery of a therapeutic.

In another embodiment, the composition is provided in the form of a patch, for example for application to the skin, or other epithelial tissue barrier. In another embodiment, the composition is provided in the form of a pessary or a suppository.

In another embodiment, the composition comprises a polymer, such as a polymer matrix or film, for example for controlled release. The agent of the invention, and optionally the payload molecule, may be encapsulated by a polymer, for example for controlled release. The agent of the invention, and optionally the payload molecule, may be impregnated in a polymer, for example for controlled release. The polymer may be a solid, or a viscous liquid (i.e. having a substantially higher viscosity than water at 25° C.). The polymer may be a gel or paste.

The composition may comprise one or more pharmaceutically acceptable excipients. The composition may comprise one or more ophthalmically acceptable ingredients selected from the group consisting of: water; saline; salt; buffer; demulcent; humectant; viscosity increasing agent; tonicity adjusting agent; cellulose derivatives e.g. carboxymethylcellulose sodium, hydroxyethyl cellulose, hydroxypropyl methylcellulose, or methylcellulose; dextran 70; gelatin; polyols; glycerine; polyethylene glycol e.g. PEG300 or PEG400; polysorbate 80; propylene glycol; polyvinyl alcohol; and povidone (polyvinyl pyrrolidone); and combinations thereof. The composition may comprise poloxamer, such as poloxamer 407 (F127™).

Demulcents may comprise or consist of cellulose derivatives, glycerine, polyvinyl alcohol, polyvinyl pyrrolidone, cellulose derivatives, polyethylene glycol, or combinations thereof.

The composition may be formulated as a nanoparticle (e.g. lipid droplet/vesicle or polymer/sol-gel based). Such formulations are known in the art. The skilled person will appreciate that targeted delivery can be achieved by using such a formulation which will then degranulate at a particular location, allowing systemic delivery but localised activity.

### Medical Uses

The composition according to the invention can be provided for use in medicine.

In one embodiment, it may be provided for use in the treatment or prevention of an eye disorder in a subject.

Accordingly, there is provided the use of a composition according to the invention herein in the manufacture of a medicament for treatment or prevention of an eye disorder in a subject.

There is also provided a method of treatment or prevention of an eye disorder in a subject comprising the administration of the composition according to the invention to an eye of the subject.

The administration may be topical, to the surface of the eye or to the eyelid.

The eye disorder may be selected from macular degeneration, cancer, diabetic retinopathy, retinal vascular occlusive disease, inflammatory eye disease and retinopathy of prematurity.

In one embodiment, the eye disorder comprises macular degeneration, such as AMD (age-related macular degeneration). The AMD may be wet-AMD or dry-AMD. The eye disorder may comprise diabetic retinopathy. In another embodiment, the eye disorder may comprise cancer, such as a solid tumour cancer, melanoma, and retinoblastoma. In another embodiment, the eye disorder comprises diabetic retinopathy. In another embodiment, the eye disorder comprises retinal vascular occlusive disease, such as venous stasis retinopathy, central retinal vein occlusion, and branch retinal vein occlusion. In another embodiment, the eye disorder comprises inflammatory eye disease, such as iritis, uveitis, scleritis, blepharitis and orbital inflammatory disease. In another embodiment, the eye disorder comprises retinopathy of prematurity.

The eye disorder may be acute or chronic.

There is also provided a composition according to the invention herein for use in the treatment or prevention of a respiratory disorder in a subject.

There is also provided the use of a composition according to the invention herein in the manufacture of a medicament for treatment or prevention of a respiratory disorder in a subject.

There is also provided a method of treatment or prevention of a respiratory disorder in a subject comprising the administration of the composition according to the invention to the lung of the subject.

The respiratory disorder may be selected from microbial infection (e.g. viral or bacterial), cancer, asthma, interstitial lung disease, inflammatory lung disease, tumours and COPD.

In one embodiment, the respiratory disorder comprises cancer, such as lung cancer. In another embodiment, the respiratory disorder comprises microbial infection, or associated inflammatory effects of microbial infection. In another embodiment, the respiratory disorder comprises viral infection. The viral infection may be coronavirus infection, such as SARS-CoV-2 (Covid-19), or influenza. In another embodiment, the microbial infection is a bacterial infection, such as Bacillus anthracis. In another embodiment, the microbial infection is a fungal infection. The fungal infection may comprise aspergillus. In another embodiment, the respiratory disorder comprises inflammatory disorder, such as asthma. In one embodiment, the respiratory disorder comprises COPD.

The respiratory disorder may be acute or chronic.

There is also provided a composition according to the invention herein for use in the treatment or prevention of a GI tract disorder in a subject.

There is also provided the use of a composition according to the invention herein in the manufacture of a medicament for treatment or prevention of a GI tract disorder in a subject.

There is also provided a method of treatment or prevention of a GI tract disorder in a subject comprising the administration of the composition according to the invention to the GI tract of the subject.

The GI tract disorder may be selected from inflammatory bowel disease, such as Crohn's disease and ulcerative colitis, cancer, such as bowel cancer, oesophageal cancer, mouth cancer, tongue cancer, or stomach cancer.

The GI tract disorder may be acute or chronic.

The composition may be formulated for oral administration. The composition may comprise one or more orally acceptable excipients.

There is also provided a composition according to the invention herein for use in the treatment or prevention of a skin disorder in a subject.

There is also provided the use of a composition according to the invention herein in the manufacture of a medicament for treatment or prevention of a skin disorder in a subject.

There is also provided a method of treatment or prevention of a skin disorder in a subject comprising the administration of the composition according to the invention to the skin of the subject.

The skin disorder may be selected from skin neoplasms and inflammatory skin disorders. In one embodiment the skin disorder comprises a skin neoplasm, which may be cancerous. The cancerous skin neoplasm may be a cancer, such as skin cancer (e.g. melanoma), or solid tumours presenting in the dermis or subcutaneous tissue. In another embodiment, the skin disorder comprises inflammatory skin disorders, such as eczema, acne and psoriasis.

The administration may be topical to the surface of the skin. The administration may be via a transdermal patch or gel.

There is also provided a composition according to the invention herein for use in the treatment or prevention of a brain disorder, such as brain cancer, in a subject.

The use may be in a method of treatment wherein the composition is delivered systemically or nasally to the subject, for transport of the payload molecule across the blood brain barrier.

There is also provided a method of treatment or prevention of a brain disorder, such as brain cancer, in a subject comprising the systemic of nasal administration of the composition according to the invention to the subject.

There is also provided a composition according to the invention herein for use as a vaccine.

There is also provided a method of vaccination, the method comprising administration of the composition according to the invention herein to a subject as a vaccine.

The use as a vaccine may be for the prevention or treatment of a disease in a subject. The disease may be an infectious disease, such as a viral or bacterial infectious disease. In another embodiment, the disease may be cancer.

In general, when considering the treatment or prevention of a medical condition, the administration of the composition may be a pharmaceutically effective amount of the composition. The treatment or prevention may comprise a single administration or repeated administrations. The administration may be once every 1 to 18 days, or more. The administration may be once every 5 to 18 days, or more. The administration may be once every 7 to 18 days, or more. The administration may be once every 10 to 18 days, or more. The administration may be once every 15 to 18 days, or more. The administration may be about once every 7 days or once every month. The administration may be once every 1 to 30 days. The administration may be no more than once per day. In one embodiment, the administration is weekly, monthly, or every 2-3 weeks.

### Products and Kits

The invention provides products that comprise composition according to the invention herein.

There is provided an eye drop dispenser or eye wash device comprising the composition according to the invention herein.

An eye drop dispenser may otherwise be known as an eye drop applicator. Typical eye drop dispensers comprise a reservoir for the composition and an outlet for the composition. The outlet may be tapered towards a distal end, with the outlet orifice at the tip/distal end. The dispenser may be arranged to be sealed, for example with a cap. An eye drop dispenser may alternatively comprise a syringe device. An eye drop dispenser may alternatively comprise a spray device.

There is provided an aspirator device comprising the composition according to the invention herein. There is also provided an inhaler or a nebuliser device comprising the composition according to the invention herein.

The composition may be provided in a gel or liquid form, suitable for administration through bronchoscopy.

There is also provided a controlled release tablet comprising the composition according to the invention herein.

There is also provided a transdermal patch or gel comprising the composition according to the invention herein.

Thus, in one embodiment, there is provided a patch, such as a saline-soaked patch, containing the composition according to the invention. Such a patch may provide a recording portal into the subject's body. The patch may be applied to the epithelial barrier, such as the skin. The patch may be applied underneath a smart watch or other personal electronic monitoring device. The monitoring could be the monitoring of acute or chronic conditions. In one embodiment, the monitoring may be for electrophysiological signals, biomarker or analyte concentrations, alone or in combination.

There is also provided a kit comprising:
a payload molecule; and
an agent capable of disrupting the epithelial tissue barrier.

The agent is as defined and described above. The payload molecule may be as defined and described above.

The payload molecule and agent capable of disrupting the epithelial tissue barrier may be formulated in separate compositions or dispensers. The payload molecule and agent capable of disrupting the epithelial tissue barrier may be formulated in separate containers and arranged to be mixed prior to use. The payload molecule and agent capable of disrupting the epithelial tissue barrier may be co-formulated in a single composition or dispenser. Separate compositions may be mixed prior to use/administration, or they may be used/administered separately, such as by sequential or concurrent administration.

The kit may further comprise one or more containers for the payload molecule and compositions comprising the agent capable of disrupting the epithelial tissue barrier. The kit may further comprise an applicator, such as an eye drop dispenser, a syringe, or a transdermal patch.

### Other Uses

The agent as defined and described above may be used to facilitate penetration of a payload molecule through an epithelial tissue barrier.

The payload molecule may be co-formulated with the agent capable of disrupting the epithelial tissue barrier.

The agent as defined and described above may be used to facilitate extracellular fluid extraction from a subject, wherein the use comprises the application of the agent capable of disrupting the epithelial tissue barrier to an epithelial tissue barrier and the extraction of extracellular fluid through the epithelial tissue barrier.

A method for extracellular fluid extraction from a subject is also provided, the method comprising the application of an agent capable of disrupting the epithelial tissue barrier to an epithelial tissue barrier of the subject, and the extraction of extracellular fluid through the epithelial tissue barrier, wherein the agent capable of disrupting the epithelial tissue barrier is as defined and described above.

Extraction of extracellular fluid through the epithelial tissue barrier may be carried out by applying a vacuum to the agent-treated surface to extract the extracellular fluid.

The extracted extracellular fluid may be used for analysis, such as for electrophysiological measurements and/or biomarker analysis. Biomarkers may include glucose (e.g. for diabetes control), proteins, volatiles/gases, acidosis/pH/ion balance and/or exosomes (such as up to ˜30 nm).

The present invention may be used in applications such as needle-free ink delivery (e.g. for agriculture and/or the tattoo industry); needle-free delivery of therapeutics, such as vaccines (e.g. covid vaccines); targeted delivery of therapeutics to the lung via aerosols; targeted delivery of therapeutics to the brain, such as via the nasal epithelial barriers; long-term deliveries, such as chemotherapies; or ocular delivery in a contact lens or contact lens solution. In particular, the payload may be a molecule or therapeutic for use in such applications. In one embodiment, the composition according to the present invention is used for needle-free delivery of a therapeutic agent, for example through the skin.

The composition may be formulated in a nebuliser/atomiser or a vape for delivery of payload through epithelial barriers in the respiratory tract.

In an embodiment wherein the invention is used for a tattoo, it can provide a painless subcutaneous delivery of pigments, particles or macromolecules, which may be patterned by painting, rolling, spraying, stamping, direct printing, or screen-printing.

Fluorescent pigments and particles may be the payload delivered through or into the skin. The payload may include quantum dots, nanoparticles, non-fading Au nanoparticle-based colours or other macromolecules.

The invention may be used for oedema release or peripheral drainage. In such embodiments, the skilled person will recognise that the composition of the invention may not include a payload for delivery.

The invention may also be used in applications requiring the measurement of bioelectric signals, which may be recorded from a subject. For example, the epithelial barrier disruption can reduce transepithelial resistance to improve the signal to noise ratio of bioelectric signals, such as electroencephalogram (EEG), electrocardiogram (ECG) and electroretinogram (ERG) signals.

Therefore, according to another aspect of the present invention, there is provided a method of measuring a bioelectric signal from a subject, the method comprising the administration of the composition according to the invention to an epithelial barrier surface, such as the skin, and measuring electric signals therefrom with an electrode. The composition according to the invention may be applied to the epithelial barrier surface, such as the skin, in the form of a gel or a patch (e.g. an adhesive patch). Electrodes capable of detecting the electric signals can be applied to the epithelial barrier surface, such as the skin, before the composition, at the same time as the composition, or after the composition. In one embodiment the electrodes may be attached using the composition when provided in the form of a patch. In one embodiment the composition is provided in the form of a gel and the electrodes are applied with the gel or after the gel.

### Definitions

The term “prevention” means avoidance of a disorder or a protective treatment for a disorder. The prevention may include a reduced risk of the disorder, reduced risk of infection, transmission and/or progression, or reduced severity of the disorder.

The term “treatment” means a cure of a condition or disease, an alleviation of symptoms, or a reduction in severity of a disorder or symptoms of the disorder.

Reference to “solid tumour” herein is intended to refer to an abnormal mass of tissue or growth in a subject's body. Solid tumours may be benign (not cancer), or malignant (cancer).

By “antibody” we include substantially intact antibody molecules, as well as chimeric antibodies, human antibodies, humanised antibodies (wherein at least one amino acid is mutated relative to the naturally occurring human antibodies), single chain antibodies, bispecific antibodies, antibody heavy chains, antibody light chains, homodimers and heterodimers of antibody heavy and/or light chains, single-domain antibodies, nanobodies, and antigen binding fragments and derivatives of the same. We also include antibody mimetic binding proteins, such as SoloMER™ proteins within the meaning of the term “antibody”. SoloMERs™ are a small (11 kDa) and stable proteins, similar to single-domain antibodies, but that have a fourth binding loop in the single binding domain format. In particular, the term “antibody” as used herein refers to immunoglobulin molecules and immunologically active portions of immunoglobulin molecules, i.e., molecules that contain an antigen binding site that specifically binds an antigen, whether natural or partly or wholly synthetically produced. The term also covers any polypeptide or protein having a binding domain which is, or is homologous to, an antibody binding domain. These can be derived from natural sources, or they may be partly or wholly synthetically produced. Examples of antibodies are the immunoglobulin isotypes (e.g., IgG, IgE, IgM, IgD and IgA) and their isotypic subclasses; fragments which comprise an antigen binding domain such as Fab, scFv, Fv, dAb, Fd; and diabodies. Antibodies may be polyclonal or monoclonal. A monoclonal antibody may be referred to as a “mAb”.

It has been shown that fragments of a whole antibody can perform the function of binding antigens. Examples of binding fragments of the invention are (i) the Fab fragment consisting of VL, VH, CL and CHI domains; (ii) the Fd fragment consisting of the VH and CHI domains; (iii) the Fv fragment consisting of the VL and VH domains of a single antibody; (iv) the dAb fragment which consists of a VH domain; (v) isolated CDR regions; (vi) F(ab′)2 fragments, a bivalent fragment comprising two linked Fab fragments; (vii) single chain Fv molecules (scFv), wherein a VH domain and a VL domain are linked by a peptide linker which allows the two domains to associate to form an antigen binding site; (viii) bispecific single chain Fv dimers (PCT/US92/09965, incorporated herein by reference) and; (ix) “diabodies”, multivalent or multispecific fragments constructed by gene fusion (WO94/13804, incorporated herein by reference).

The skilled person will understand that optional features of one embodiment or aspect of the invention may be applicable, where appropriate, to other embodiments or aspects of the invention.

Embodiments of the invention will now be described in more detail, by way of example only, with reference to the accompanying drawings and worked examples.

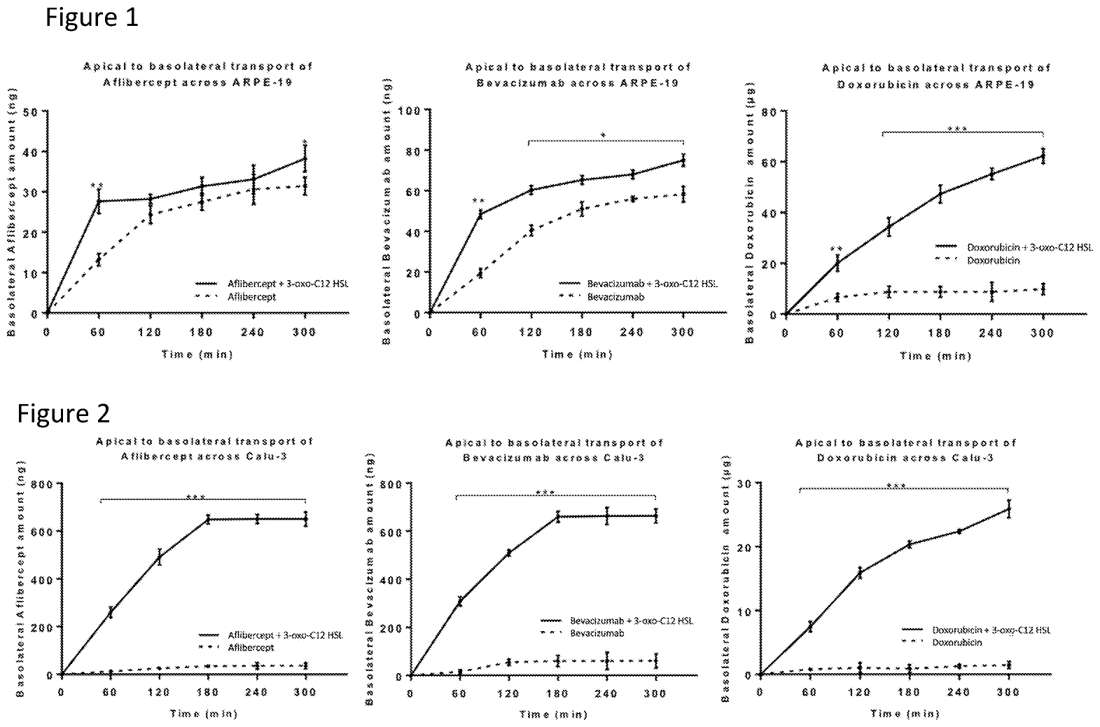

FIG. 1: Macromolecular therapeutics transported across ARPE-19 cell layers in the absence and presence of 3OC12-HSL transported into the basolateral chamber over a period of five hours. Permeability experiments were initiated by applying 0.5 ml of the test solutions (250 μg/ml Aflibercept/Bevacizumab/or Doxorubicin diluted in HBSS/HEPES) to the apical side of the cells (donor compartment of the Transwell) with or without 3OC12-HSL (200 μM). Drug permeability was determined by sampling the basolateral solution at regular time intervals. Data points are expressed as means±SEM, derived from three replicates in triplicate. p values of <0.05 were considered statistically significant. *, ** and *** denote p<0.05, p<0.01 and p<0.001, respectively.

FIG. 2: Macromolecular therapeutics transported across Calu-3 cell layers in the absence (dashed line) and presence (line) of 3OC12-HSL transported into the basolateral chamber over a period of five hours. Permeability experiments were initiated by applying 0.5 ml of the solutions (250 μg/ml Aflibercept/Bevacizumab/or Doxorubicin diluted in HBSS/HEPES) to the apical side of the cells (donor compartment of the Transwell) with or without 3OC12-HSL (200 μM). Drug permeability was determined by sampling the basolateral solution at regular time intervals. Data points are expressed as means±SEM, derived from three replicates in triplicate. p values of <0.05 were considered statistically significant. *, ** and *** denote p<0.05, p<0.01 and p<0.001, respectively.

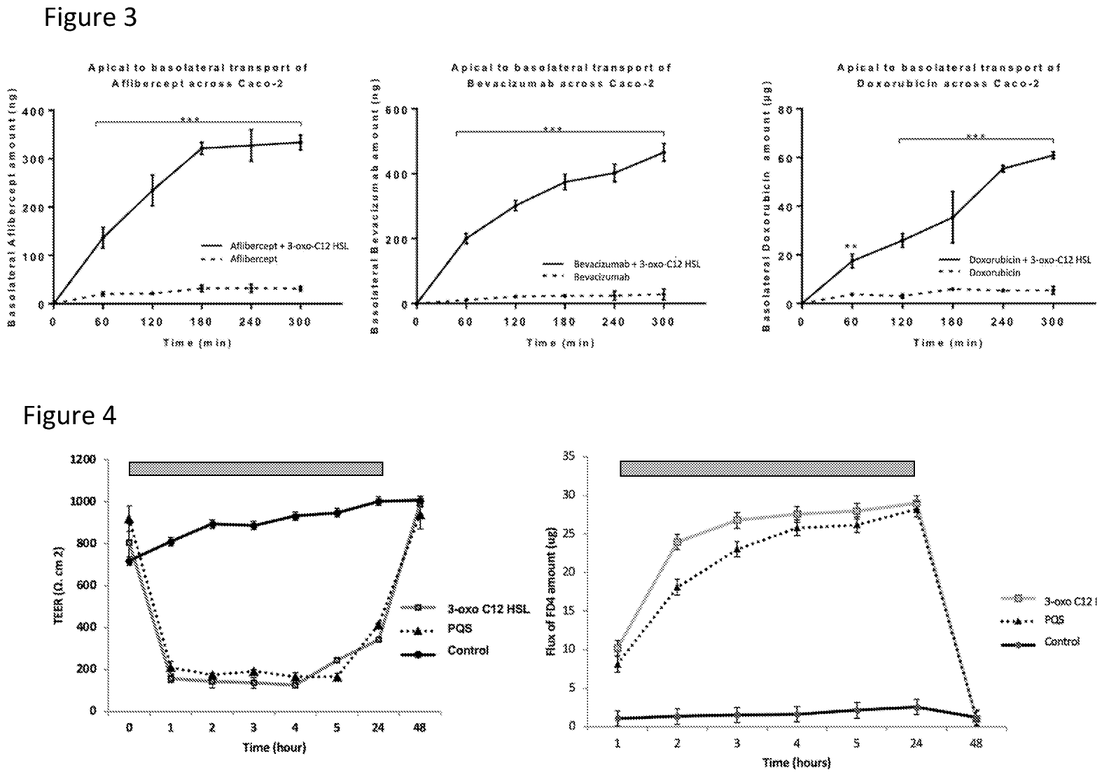

FIG. 3: Macromolecular therapeutics transported across Caco-2 cell layers in the absence (dashed line) and presence (line) of 3OC12-HSL transported into the basolateral chamber over a period of five hours. Permeability experiments were initiated by applying 0.5 ml of the test solutions (250 μg/ml Aflibercept/Bevacizumab/or Doxorubicin diluted in HBSS/HEPES) to the apical side of the cells (donor compartment of the Transwell) with or without 3OC12-HSL (200 μM). Drug permeability was determined by sampling the basolateral solution at regular time intervals. Data points are expressed as means±SEM, derived from three replicates in triplicate. p values of <0.05 were considered statistically significant. *, ** and *** denote p<0.05, p<0.01 and p<0.001, respectively.

FIG. 4: Reversible disruption of epithelial barrier function by exemplar QSSM (3-oxo-C12 homoserinelactone: “3-oxo C12 HSL”, and 2-Heptyl-3-hydroxy-4 (1H)-quinolone: “PQS”) in Transwell-cultured Calu-3 cells.

Left: Trans-epithelial electrical resistance (TEER) sampled during and after exposure (bar) using 12.5 Hz modulation (EVOM), hourly for 5 hours and thereafter at increments of 24 hr post-exposure. TEER is expressed as the measured resistance per area (cm²) of the cell layer. Background TEER due to the filter was subtracted from the reported TEER values. Data presented as the mean±SD (n=3).

Right: Transport of FITC-4 kDa dextran (FD4) across Calu-3 cell layers into the basolateral chamber following its addition to the apical chamber. Data presented as the mean±SD (n=3).

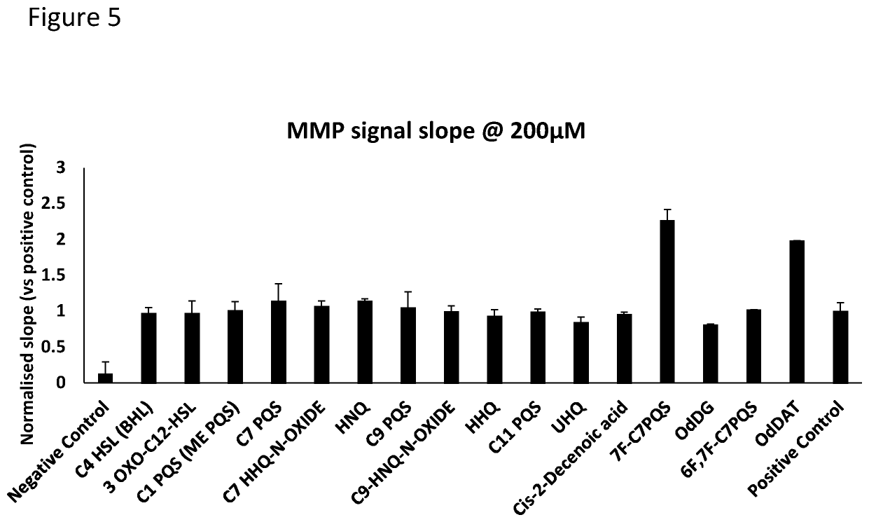

FIG. 5: High-throughput MMP assay (MMP Activity Assay Kit (Fluorometric-Red), Abcam #ab112147) showing the normalised accumulation slope of FRET-based signal (Ex540: Em590 nm) read at 5 minute intervals for one hour in adherent Calu-3 cells cultured in glass-bottomed 96-well plates using a SpectraMax i3x/Biomek FXPP high-throughput platform. Each experiment was conducted in triplicate (error bars). Signal accumulation over time in each well was linearly fitted and the gradient of each fit was scaled against the gradient of the relevant positive control wells, to which active MMP was added directly (mixed isoforms, 50 μM total MMP), to obtain the normalised accumulation slope.

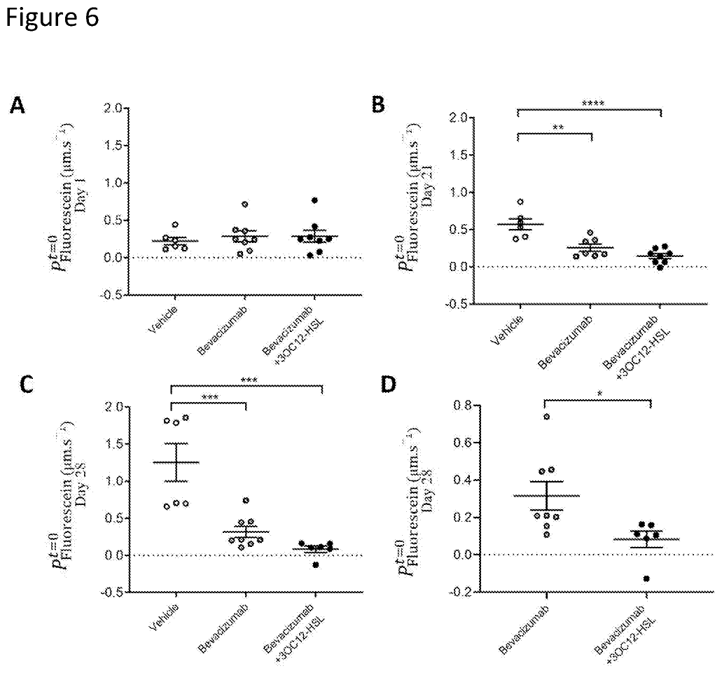

FIG. 6: Topical eye administration of Bevacizumab with and without 3OC12-HSL in a diabetic rat model with increased vascular permeability. Type I diabetes was induced in Norway Brown rats with a single i.p. injection of STZ (50 mg/kg). Control animals were administered an equivalent volume of sterile saline solution. Animals with glucose levels >15 mmol/L were deemed diabetic and included in the study. Animals received twice daily topical drops to the right eye (20 μL of bevacizumab (250 μg/mL) with/without 3OC12-HSL and control vehicle only). FFA was performed pre diabetic-induction (day 0) and on days 7, 14, 21 and 28, using a retinal ophthalmoscope. Retinal vascular permeability was calculated from the resultant angiogram avi format files and as previously described (Allen et al, 2021) All data and graphs were calculated with Fiji and GraphPad Prism v8 and statistical analysis using a one-way ANOVA, with Tukey's post-hoc test, * p=<0.05, ** p=<0.01. Data bars represent mean±SEM.

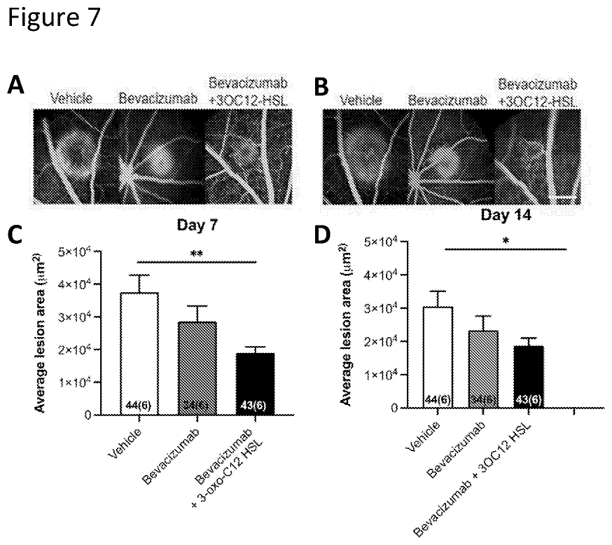

FIG. 7: Anti-angiogenic efficacy of Bevacizumab with and without 3OC12-HSL (agent) in a laser-induced mouse model of CNV.in a mouse model of choroidal neovascularisation. C57BL/6J mice (n=6 per group) were subjected to laser photocoagulation (Merilas, 532α, 450 mW, 130 ms, 75 μm, 4 lesions per eye) on day 0. Post lasering animals received twice daily topical drops to both eyes10 μL of bevacizumab (250 μg/mL) with/without 3OC12-HSL and control vehicle only) for a total of 14 days. FFA was performed on days 7 and 14 and lesion area measured using Fiji software, n=lesions (mice). All data and graphs were calculated with Fiji and GraphPad Prism v8 and statistical analysis using an t-test, with Welch's correction, * p=<0.05, ** p=<0.01. Data bars represent mean±SEM. Scale bar 200 μm.

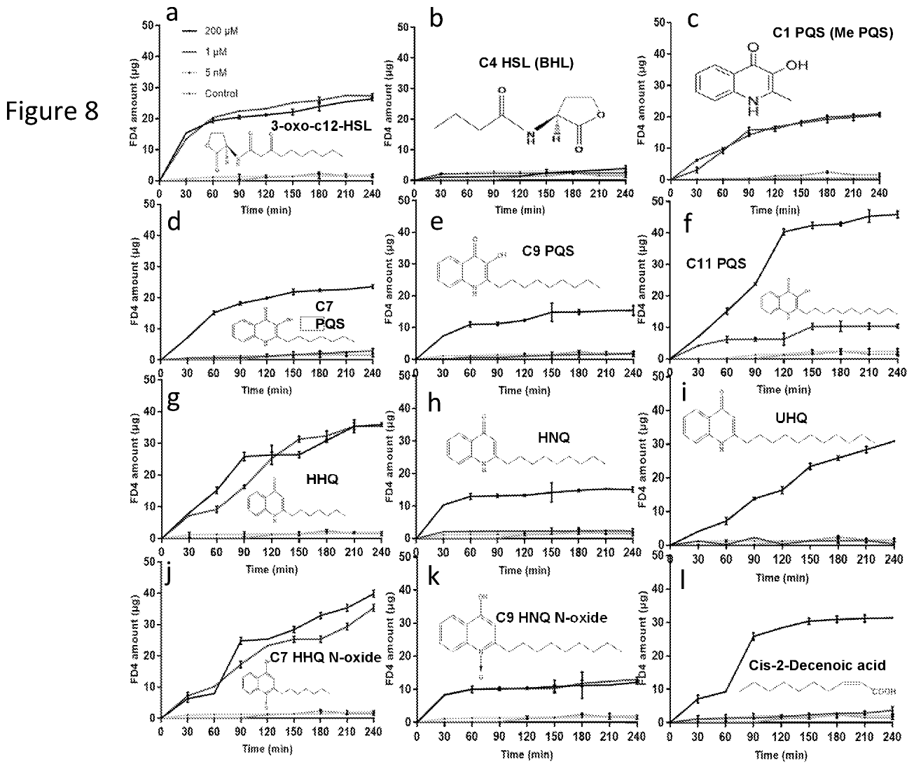

FIG. 8: Transwell confirmation of effect on FITC-dextran (4 kDa) transport. Exemplary molecules (a)-(1) investigated in coarse dose-response by FD4 delivery in Transwell culture (Calu-3). Each molecule was screened at 5 nM, 1 μM, 200 μM levels.

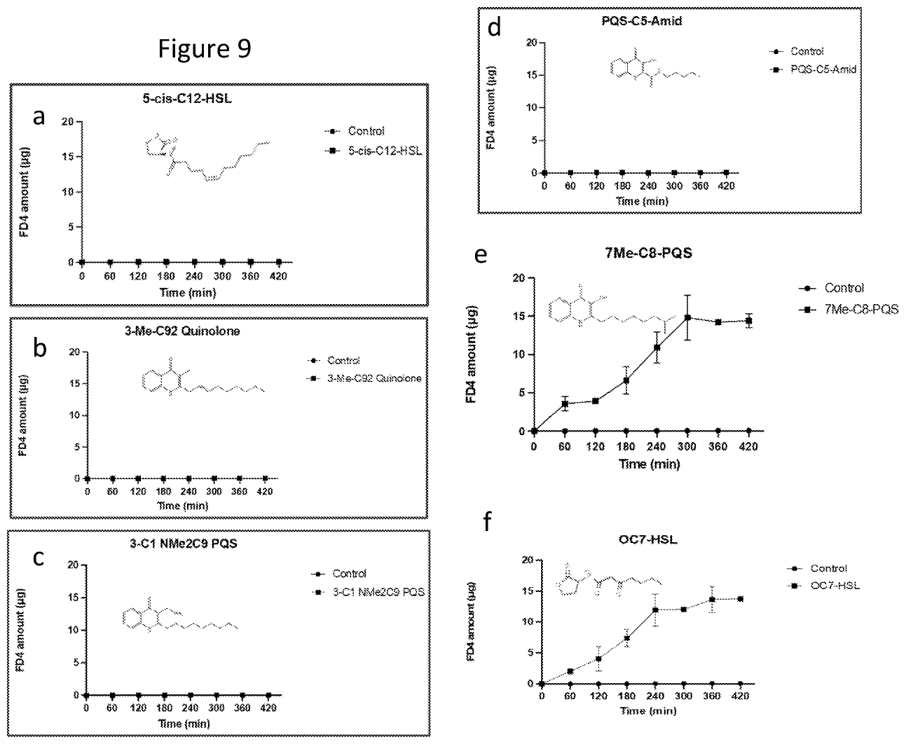

FIG. 9: Transwell confirmation of effect on FITC-dextran (4 kDa) transport. Ineffective molecules investigated in coarse dose-response by FD4 delivery in Transwell culture (Calu-3). Each compound was screened at 200 μM level. FIGS. 9a-d show molecules that are ineffective, whereas FIGS. 9e and 9f are exemplary molecules used in the present invention and these are effective.

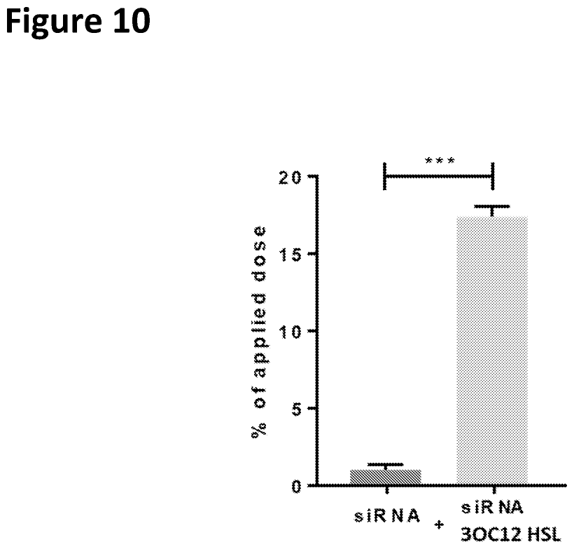

FIG. 10: Delivery of siRNA (19 bases) across Calu-3 cell layer is enhanced in the presence of 30C12 HSL (200 μM) assessed as recovered fraction from the basolateral chamber of a Transwell.

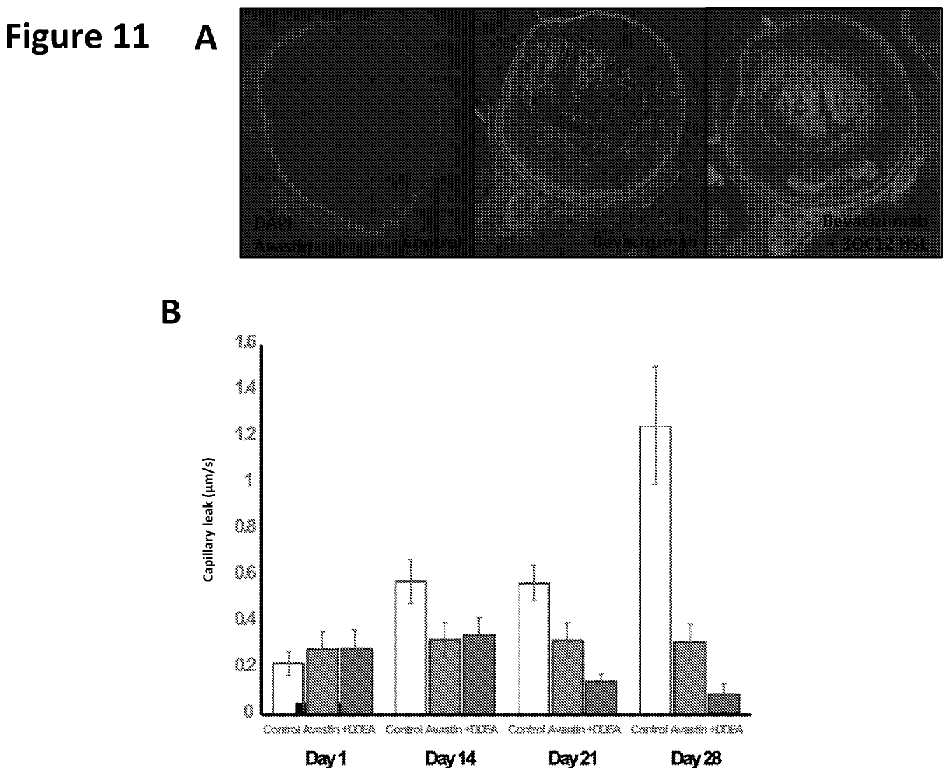

FIG. 11: A) In vivo uptake of bevacizumab into rat eye, recvered on day 28 and fixed in 4% PFA and processed for immunocytochemistry using a fluorescent anti-human IgG antibody (stains as red). B) In vivo treatment of diabetic retinopathy endpoint (capillary leak as assessed by fundus fluorescein angiography) by bevacizumab (Avastin)+30C12 HSL.

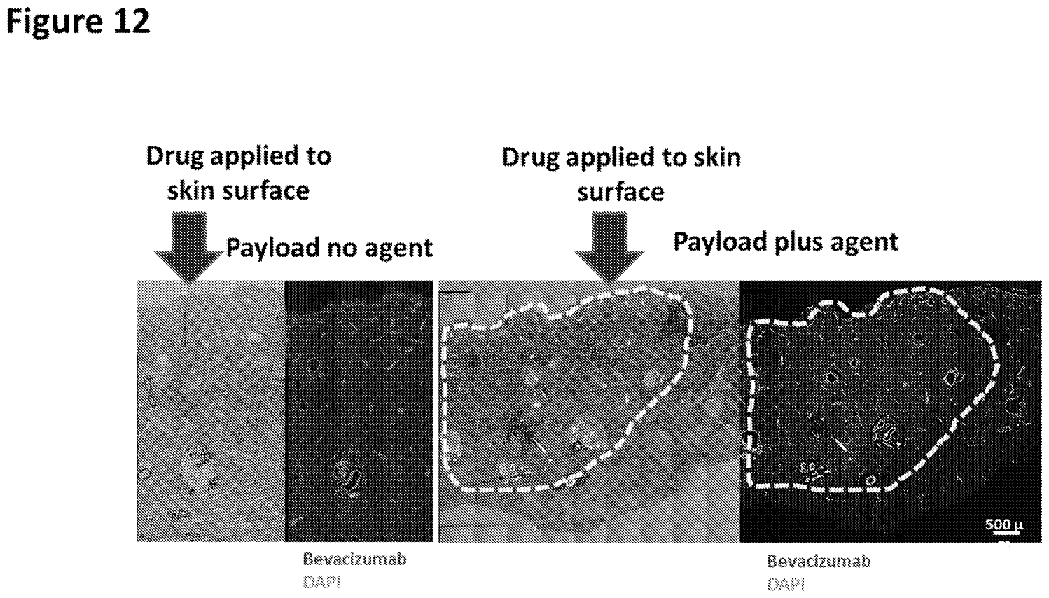

FIG. 12: Shows success in delivering large drugs through skin ex vivo with the agent 30C12 HSL. Red signal (an intense red marking is outlined by white dash lines) shows Bevacizumab delivery with 5 mm penetration in 60 minutes. All monoclonal antibodies are similar (150 kDa) and would be expected to show similar enhanced penetration. Botox is also the same size (150 kDa).

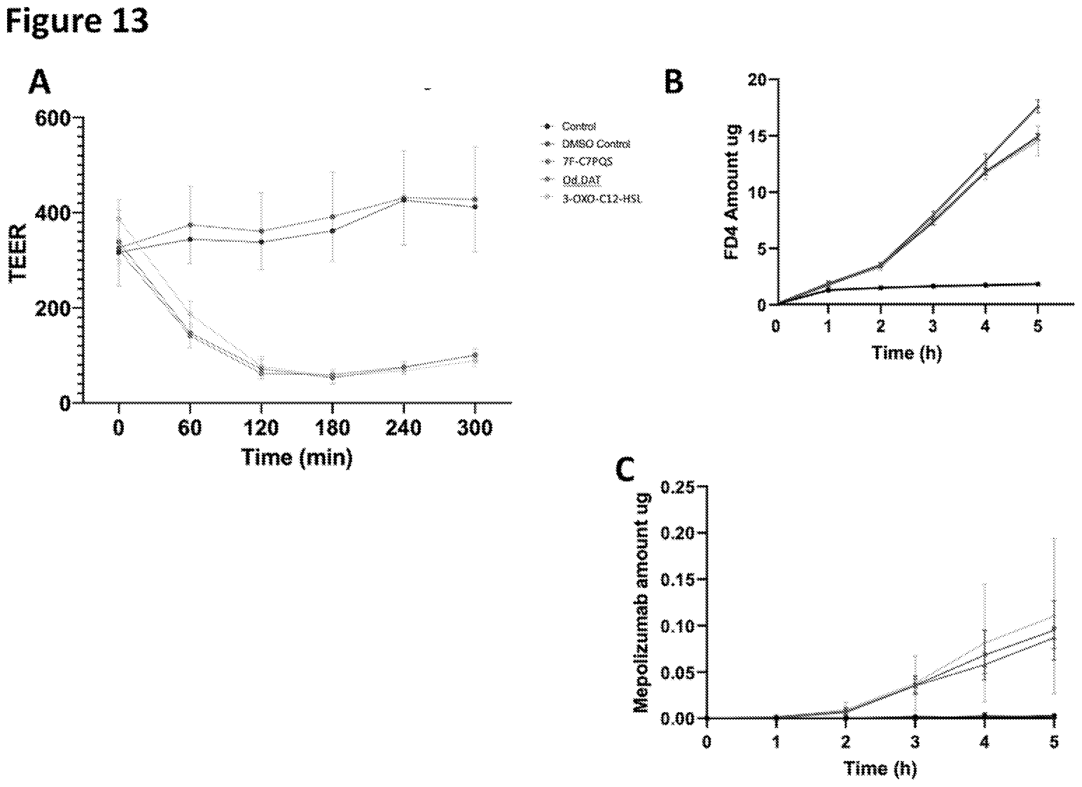

FIG. 13: Shows the effect of the three agents, 7F-C7PQS, Od.DAT, and 30C12, HSL on human primary lung epithelium (air-liquid interface culture). Upon agent exposure, TEER (electrical resistance) is temporarily reduced, FD4 delivery (permeability) is increased, and mAb (Mepolizumab) delivery is increased.

### Example A—In Vitro Studies

### Test Agents:

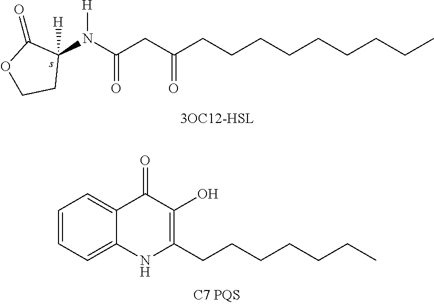 [SMILES: CCCCCCCCCC(=O)CC(=O)N[C@H]1CCOC1=O ; CCCCCCCc1[nH]c2ccccc2c(=O)c1O]

### Methods

### Cell Culture

Calu-3 cells obtained from American Type Culture Collection, USA were cultured using Dulbecco's Modified Eagle Medium (DMEM) supplemented with penicillin (100 U/mL), streptomycin (0.1 mg/mL), amphotericin (0.25 μg/mL), non-essential amino acids (NEM, 1%) and Fetal Bovine Serum (FBS, 10% v/v, Sigma-Aldrich, UK). The human-derived RPE cell line ARPE-19 was obtained from American Type Culture Collection. Cells were grown in Dulbecco's Modified Eagle Medium: F12 (1:1; Gibco, Invitrogen, Carlsbad, CA), including 10% inactivated fetal bovine serum, 100 units/mL penicillin, 100 μg/mL streptomycin, and 2 mM L-glutamine (Sigma-Aldrich). Caco-2 cells were grown in complete culture medium (DMEM supplemented with 17% FBS and 1% antibiotic-antimycotic solution). All cells were kept in a water-jacketed incubator (model 3546, Forma Scientific, Inc., Marietta, OH, USA) maintained at 37° C. and an atmosphere of humidified air with 5% CO₂. Medium was replaced every other day until cells reached the 80-90% confluence desired for re-plating or culturing mature, polarised barrier models.

When confluent, cells were seeded on Transwell® filters (12 mm diameter, 0.4 μm pore size; Corning Life Sciences, Holland) at 10⁵ cells/cm². Cell confluency and cell layer integrity were confirmed by TEER using an EVOM (World Precision Elements, USA) voltohmmeter. Cells were used only if their TEER was stable and above 600 Ω/cm² (For Calu-3 and Caco-2). Filter-cultured Calu-3 cell layers were used on day 13-14 days and Caco-2 and ARPE-19 cell layers were used on day 21 post-seeding.

### Effect of QS Molecules on Calu-3 Metabolic Activity: MTS Assay

To assess 3OC12-HSL and C7 PQS molecules cytotoxicity on cell viability of Calu-3 cell layers and to monitor short-term cytotoxicity during experiments, Calu-3 cells were treated with 3OC12-HSL and C7 PQS, at concentrations 5 nM, 1 μM, 50 μM, 100 μM and 200 μM, for 6 hours. At the end of the treatment, cell viability (through measuring metabolic activity) of cultures was determined by chromogenic MTS assay.

### Assay:

The viability of Calu-3 cells was determined by a chromogenic MTS method based on the measuring of mitochondrial respiration, assessed by the reduction of 3-(4,5-dimethylthiazol-2-yl)-5-(3-carboxymethoxyphenyl)-2-(4-sulfophenyl)-2H-tetrazolium to formazan. Calu-3 cells were cultured in a 96-well plate (CellTiter 96® Aqueous, Promega) at a density of 10⁴ cells per well and incubated at 37° C., 5% CO₂ for 24 hours. After 24 hours, the medium was replaced with 100 μl of test compounds dissolved in HBSS and then cells were incubated for 6 hours. HBSS and Triton X-100 (0.1% v/v in HBSS) were used as the negative and positive controls respectively. Bathing solutions were then aspirated and cells washed with PBS. Then culture medium (100 μl at 37° C.) plus MTS reagent (20 μl) were added into each well and cells were incubated for 2 hours. Absorbance at 492 was measured using a Dynex absorbance microplate reader (Dynex Technologies, USA) and converted into a percentage metabolic rate.

### Fluorescein Isothiocyanate-Labelled Dextran 4400 (FD4) Permeability

Culture medium was removed from filter-cultured Calu-3 cells and replaced with Hank's Balanced Salt Solution (HBSS), buffered with 2-[4-(2-hydroxyethyl) piperazin-1-yl]ethanesulfonic acid (HEPES, 20 mM); cell layers were incubated with HBSS for 45 min. Quorum sensing molecules 3OC12-HSL and C7 PQS and Dextran-FITC 4 kDa (FD4: 500 μg/ml [125 μM]) diluted in HBSS/HEPES were applied to the apical chamber of each Transwell®.

Apical-to-basolateral translocation of FD4 was determined by sampling from the basolateral side at 100 μL every 30 min over 5 hours. In order to maintain the basolateral volume constant, 100 μl of HBSS was added to the basolateral chamber after taking each sample. FD4 fluorescence (485 nm excitation, 535 nm emission) was then determined using an MFX microtiter plate fluorometer (Dynex Technologies, USA). FD4 in the basolateral solutions was quantified by converting the fluorescence readings into FD4 concentrations and amounts, through construction of calibration curves.

TEER of each cell layer was also measured at the same time points throughout the experimental period in order to assess tight junction integrity.

### Immunostaining

To visualise the effect of 3OC12-HSL and C7 PQS on tight junction integrity in Calu-3 cell layers, ZO-1, immunostaining and confocal microscopy was performed on cell layers treated by 200 μM 3OC12-HSL and C7 PQS and control (0.15% DMSO in HBSS) for one hour. Confluent cell monolayers were washed with PBS and fixed in 4% paraformaldehyde in PBS for approximately 10 min at room temperature. Cells were then washed 3 times with PBS and permeabilised by incubating with Triton X-100 (0.1% v/v in PBS) for 10 min. Cells were then washed with PBS, followed by the application of 1% BSA/PBS for approximately 1 hour. Thereafter, BSA/PBS solution was aspirated and replaced with mouse, anti-human ZO-1 (primary) antibody, diluted in 1% BSA/PBS to a final concentration of 100 μg/ml. Cell samples were incubated with the primary antibody for 2 hours. The primary antibody solution was then removed and cells washed with PBS (5 times). FITC-labelled goat, anti-mouse (secondary) antibody, diluted according to manufacturer's instructions in 1% BSA/PBS was then applied to the cells for 1 hour. The secondary antibody solution was then aspirated and cells washed with PBS three times. The Transwell® filter membrane was excised and mounted on glass slides (using DAPI-containing, ProLong® Gold antifade/mounting medium) and covered with a glass cover slip for confocal imaging, which was performed using a Zeiss LSM 880 confocal microscope.

### Effect of 3OC12-HSL on Drug Permeability In-Vitro

Three polarised epithelial in-vitro tissue models, including lung epithelium (Calu-3), retinal pigment epithelium (ARPE-19) and intestinal epithelial (Caco-2) were used to study the efficiency of 200 μM 3OC12-HSL as a paracellular permeability enhancer for three different drugs: bevacizumab, aflibercept and doxorubicin (applied concentrations 250 μg/mL). The amount of drugs transported was determined via the conversion of fluorescence intensity to amount (μg or ng), using calibration curves constructed via serial standard dilutions.

### Transport Studies

Prior to drug delivery experiments the culture medium was removed, cells were washed with PBS, and HBSS/HEPES (warmed to 37° C.; pH 7.4) was then added to both sides (apical and basolateral) of the cell monolayer. Cells were equilibrated in HBSS/HEPES (i.e. incubated at 37° C., 5% CO2) for approximately 45 min prior to experiments.

Permeability experiments were initiated by applying 0.5 mL of the test solutions to the apical side of the cells (donor compartment of the Transwell®). Test solutions contained drugs (250 μg/mL bevacizumab, aflibercept or doxorubicin diluted in HBSS/HEPES) with or without 3OC12-HSL (200 μM), which was investigated for its permeability-enhancing effect. Drug permeability was determined by sampling the basolateral solution at regular time intervals. Specifically, 100 μl volumes were removed from the basolateral chamber of the Transwell® system every 60 min for 5 hours. The sampled solutions were immediately replaced with equal volumes of the transport medium (HBSS).

### Quantitation of Bevacizumab and Aflibercept by ELISA Following Transport Experiments

Solutions (100 μl volume), sampled during bevacizumab and aflibercept transport experiments were placed in high-binding 96-well ELISA plates and incubated overnight at 4° C., so that any IgG present in the sample solutions coated the surfaces of the multi-well plate. The following day, sample solutions are removed and the plate is washed three times with the wash buffer, containing Tween20, 0.05% v/v in PBS. The wells were then blocked by 200 μl of 1% BSA w/v in PBS (blocking buffer) for 1 hour at room temperature. Blocking buffer is then removed and the wells washed with the wash buffer). 100 μl of mouse anti-human IgG conjugated to horseradish peroxidase (HRP), diluted in blocking buffer, is added to each well. Plates were incubated for 2 hours at room temperature, labelling both bevacizumab and aflibercept in each well via the IgG Fc domain. Anti-human IgG-HRP solution was then removed and the plate wells washed 3× with wash buffer. Chromogenic TMB substrate (100 μl) was added into each well for 10 min before the reaction is stopped by adding 20 μl of 2.5 M sulphuric acid, following which the plates are transferred into a microplate reader for determination of absorbance (at 450 nm) of the HRP-converted product. IgG, and therefore macromolecular therapeutic, concentration was quantified by converting the absorbance readings into concentrations through complementary calibration curves, created through serial dilution.

### Quantitation of Doxorubicin

Apical-to-basolateral translocation of intrinsically-fluorescent doxorubicin was determined by fluorescence plate reader (λₑₓ 470 nm; λₑₘ 585 nm) using MFX microtiter plate fluorometer (Dynex Technologies, USA).

### Statistical Analysis

All experiments were carried out using triplicate samples and were repeated three times. One-way analysis of variance (ANOVA) followed by Bonferroni post-hoc test was applied for comparison among group means of three or more groups, whilst Student's t-test was used for comparison of two groups. Data shown as the mean±SEM of three different experiments. p values of <0.05 were considered statistically significant. *, ** and *** in figures denote p<0.05, p<0.01 and p<0.001, respectively.

Effect of 3OC12-HSL and C7 PQS on the Paracellular Flux of FITC-Dextran (4 kDa) (FD4) and Transepithelial Electrical Resistance (TEER) of Polarised Calu-3 Cell Layers

To study the impact of 3OC12-HSL and C7 PQS on barrier function of airway epithelia, human-derived Calu-3 cell layers were treated with 3OC12-HSL and C7 PQS, and the effects on barrier function was determined by measuring TEER and monitoring the trans-barrier transport of FD4 across mature Calu-3 cell layers.

### Results

### Effect of 3OC12-HSL on Drug Permeability In-Vitro

FIGS. 1-3 show the amount of drugs present in the basolateral solution at different times following their addition to the apical surface of the Transwell model tissue barrier.

The translocation of all drugs across epithelial cell models increased significantly when applied with 3OC12-HSL, in comparison with the same drugs applied in the absence of 3OC12-HSL.

Doxorubicin transport was enhanced by 3OC12-HSL 6-fold in ARPE-19 (62.33 μg vs 9.85 μg) (FIG. 1), 17-fold in Calu-3 (25.90 μg vs 1.45 μg) (FIG. 2) and 19-fold in Caco-2 (59.33 μg vs 3.07 μg) (FIG. 3) cell layers after 5 hours. These enhancements in delivered therapies were statistically significant at the α=0.05 level.

Bevacizumab transport also enhanced significantly in the presence of 3OC12-HSL across Calu-3 (10-fold: 663.43 ng vs 61.03 ng) (FIG. 2) and Caco-2 (8-fold: 469.22 ng vs 54.64 ng) (FIG. 3) cell layers. Bevacizumab permeability was increased significantly across ARPE-19 cell layers (FIG. 1) by 2.5-fold at 60 minutes of exposure (** p<0.01).

Aflibercept transport was significantly (*** p<0.001, n=3) enhanced by 3OC12-HSL across Calu-3 layers (18-fold: from 36.21 ng to 650.34 ng) (FIG. 2) and Caco-2 cell layers (11-fold: from 33.82 ng to 376.27 ng) (FIG. 3) indicating that the opening of the paracellular pathway was efficient in these layers.

### Cytotoxicity: MTS Assay

No significant reduction in cell metabolic activity was observed. Cell viability of >75% was retained in all cases. Thus, 3OC12-HSL and C7 PQS do not have an adverse cytotoxic effect on cell viability within epithelial layers. Specifically, 200 μM 3OC12-HSL did not change the metabolic activity in all three cell lines compared to the negative control indicating that this high concentration of 3OC12-HSL was well tolerated by cells.

Effect of 3OC12-HSL and C7PQS on the Paracellular Flux of FITC-Dextran (4 kDa) (FD4) and Transepithelial Electrical Resistance (TEER) of Polarised Calu-3 Cell Layers

Treatment of the Calu-3 cell layer with 3OC12-HSL (at a concentration of 200 μM or 1 μM in 0.15% DMSO in HBSS) reduced TEER significantly (p<0.001), compared to vehicle only (0.15% DMSO in HBSS). This TEER reduction was correlated with the increased paracellular passage of FD4 across cell layers.

The flux of FD4 peaked after 30 minutes and continued increasing slowly for four hours (duration of the study period).

With reference to FIG. 4, the effect of 3OC12-HSL cell layer integrity was shown to be reversible, as TEER started increasing after 4 hours and reached 520+/−20 Ω·cm² after 7 hours. At the end of the experiment, 3OC12-HSL was removed and replaced with clean cell culture medium, after which the TEER of Calu-3 cell layers returned to baseline (˜940+/−40 Ωcm⁻²) after 24 hours.

Treatment of Calu-3 cell layers with C7 PQS at a concentration of 200 μM likewise increased the flux of FD4 from the apical to basolateral chamber. It also reduced the TEER from 1200 Ωcm−2 to 98 Ωcm⁻² within the first hour of exposure. After 5 hours, the integrity of the cell layer began to recover and TEER reached 510 Ωcm⁻² (SEM+/−20) after 7 hours.

Treatment with 200 μM C7 PQS also enhanced the trans-layer flux of FD4 across Calu-3 cell layers.

### Effect of 3OC12-HSL and C7 PQS on ZO-1 Distribution

Morphological changes within tight junctions resulting from the application of 200 μM 3OC12-HSL and C7 PQS could be seen. In this regard, a continuous ring of fluorescence, arising from ZO-1 staining, at cell-cell contacts was clearly visible in control cell layers exposed only to vehicle (0.15% DMSO). In contrast, considerable dislocation of ZO-1 proteins was clearly apparent in cell layers treated with 200 μM 3OC12-HSL and PQS.

These observations, taken together with the impairment of barrier function observed in the presence of both 3OC12-HSL and C7 PQS, strongly suggests that the underlying mechanism somehow disrupts tight junction organisation.

### Example B—Further in vitro studies

A number of further molecules were also tested using the in vitro Transwell studies, to assess whether they showed the same ability to enhance translocation of drugs across epithelial cell models.

The results were as follows:

TABLE 1

| | | Effective in |
|---|---|---|
| | | enhancing |
| Agent | Structure | translocation |
| C1 PQS (Me PQS) | 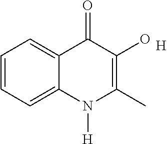 | Yes |
| UHQ | 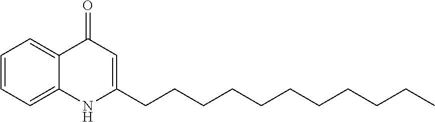 [SMILES: CCCCCCCCCCCc1cc(=O)c2ccccc2[nH]1] | Yes |
| C11 PQS | 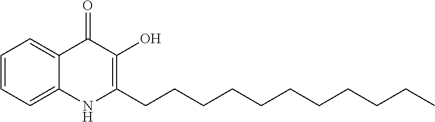 [SMILES: CCCCCCCCCCCc1[nH]c2ccccc2c(=O)c1O] | Yes |
| HHQ | 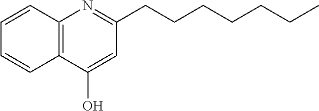 [SMILES: CCCCCCCc1cc(O)c2ccccc2n1] | Yes |
| C9 PQS | 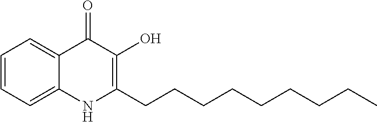 [SMILES: CCCCCCCCCc1[nH]c2ccccc2c(=O)c1O] | Yes |
| Cis-2- decenoic acid | 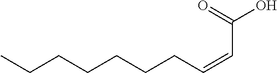 | Yes |
| C7 HNQ N-oxide | 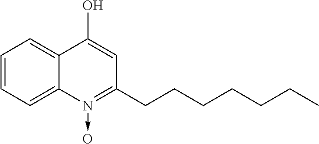 [SMILES: CCCCCCCc1cc(O)c2ccccc2[n+]1[O-]] | Yes |
| C9 HNQ N-oxide | 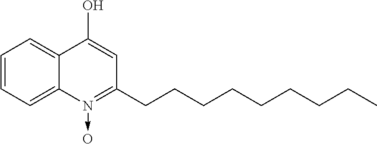 [SMILES: CCCCCCCCCc1cc(O)c2ccccc2[n+]1[O-]] | Yes |
| HNQ | 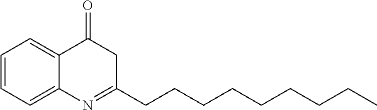 [SMILES: CCCCCCCCCC1=Nc2ccccc2C(=O)C1] | Yes |
| C7 PQS | 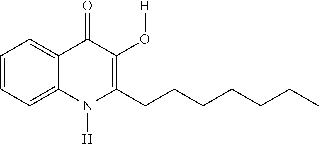 [SMILES: CCCCCCCc1[nH]c2ccccc2c(=O)c1O] | Yes |
| 3-OXO- C12-HSL | 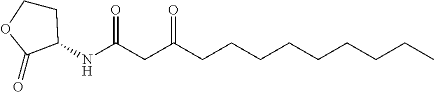 [SMILES: CCCCCCCCCC(=O)CC(=O)N[C@H]1CCOC1=O] | Yes |
| 5-cis-C12 HSL | 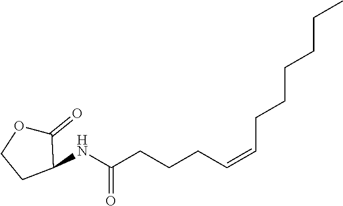 [SMILES: CCCCCC/C=C\CCCC(=O)N[C@H]1CCOC1=O] | No |
| 7Me-C8- PQS | 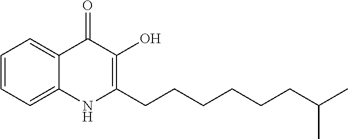 [SMILES: CC(C)CCCCCCc1[nH]c2ccccc2c(=O)c1O] | Yes |
| PQS-C5- Amide | 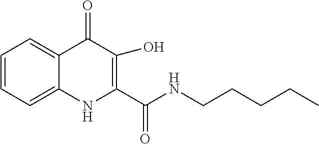 [SMILES: CCCCCNC(=O)c1[nH]c2ccccc2c(=O)c1O] | No |
| OC7-HSL | 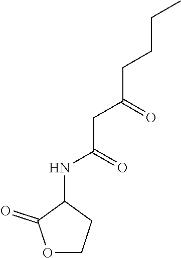 [SMILES: CCCCC(=O)CC(=O)NC1CCOC1=O] | Yes |
| 3- C1NMe2 C9 PQS | 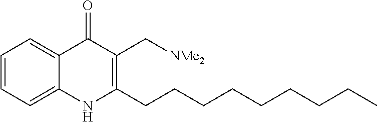 [SMILES: CCCCCCCCCc1[nH]c2ccccc2c(=O)c1CN(C)C] | No |
| 3-Me C9: 2. Quinolone | 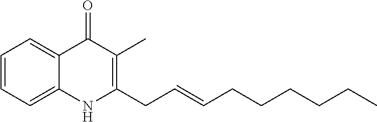 [SMILES: CCCCCC/C=C/Cc1[nH]c2ccccc2c(=O)c1C] | No |

Therefore, a range of agents in accordance with the invention have been shown to be effective in enhancing paracellular permeability for payload molecules.

The results for all of the molecules shown as “Yes” in the right-hand column were comparable to 3OC12-HSL and C7 PQS.

When looking at the molecules that were not effective (shown as “No” in the right-hand column), these failed to meet the structural requirements of the present invention due to:
the presence of a double bond or N heteroatom within the R′ chain; and/or
the presence of a —CH₂NMe substituent at the R² position (ortho to the R′ group).

### Example C—MMP Activation as Possible Screen for Activity

### MMP Activity Assay-3OC12-HSL and C7 PQS

To investigate the involvement of MMP in the disruption of tight junctions observed in the presence of 200 μM 3OC12-HSL and C7 PQS, a fluorogenic MMP activity assay was performed, using the Amplite™ Universal Fluorimetric MMP Activity Assay Kit. This kit is designed to check the general activity of a MMP enzyme.

Following treatment with 3OC12-HSL and C7 PQS respectively, the conditioned media were collected every hour from apical chamber and MMP activity was determined by using Amplite™ Universal Fluorimetric MMP Activity Assay Kit Green Fluorescence according to the manufacturer's instructions.

Review of the samples as taken at one-hour intervals from apical chamber during QS exposure, revealed significant MMP activation (p<0.01) in the samples treated with 30C12-HSL and in the samples treated with C7PQS.

No fluorescence was observed in control samples.

The MMP activation could be seen to increase over the first 4 hours for both 3OC12-HSL and C7 PQS. Then for samples taken after 4 hours, the MMP activation could be seen to start to reduce.

This correlation in time with the observed fall in TEER and increase in transport of FD4 strongly implies that MMP activation by QS molecules underlies the observed disruption in barrier function.

### Further MMP Screening

A fluorogenic MMP activity assay was carried out in the same manner on further agents.

The following further agents were found to cause MMP activation and therefore are believed to be useful as agents according to the invention.

TABLE 2
further agents causing MMP activation

| | |
|---|---|
| 7F-C7 PQS | 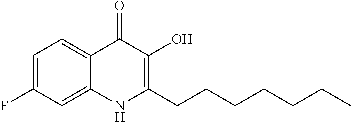 [SMILES: CCCCCCCc1[nH]c2cc(F)ccc2c(=O)c1O] |
| 6F,7F-C7 PQS | 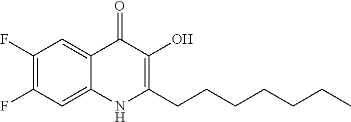 [SMILES: CCCCCCCc1[nH]c2cc(F)c(F)cc2c(=O)c1O] |
| OdDAT | 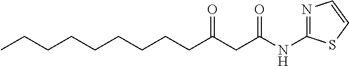 [SMILES: CCCCCCCCCC(=O)CC(=O)Nc1nccs1] |
| OdDG | 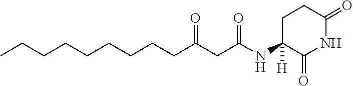 [SMILES: CCCCCCCCCC(=O)CC(=O)N[C@H]1CCC(=O)NC1=O] |

### Further Testing:

C4 HSL (BHL) was also tested and was found to cause MMP activation and therefore is also believed to be useful as an agent according to the invention:

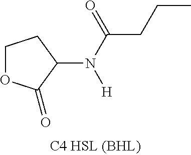

FIG. 5 shows agents causing MMP activation, with a negative control and a positive control for reference.

### Example D: Ex Vivo Studies

Ex vivo studies were carried out to confirm that the beneficial effect on epithelial barriers, as shown in vitro, was also replicated ex vivo.

3OC12-HSL was used as an exemplary agent for these studies.

### Effect of 3OC12-HSL on Drug Permeability Ex-Vivo

In order to investigate the efficacy of 3OC12-HSL as drug delivery enhancer in intact tissue, bevacizumab (250 μg/mL: 167 μM) and aflibercept (40 mg/mL: 348 μM) were applied with and without 3OC12-HSL to ex-vivo rat eyes, and the distribution of drugs was visualised within sagittal ocular cryosections by confocal microscopy. A further set of experiments tested efficacy of 3OC12-HSL via ex vivo porcine skin.

### Ex-Vivo Studies

Six hooded rats with pigmented eyes, weighing 280-300 g and aged 7-8 weeks were provided by Bio-Support Unit at the Queen's Medical Centre, Nottingham. Experiments were designed to be performed ex-vivo, immediately post-sacrifice, during experiments supporting other studies. Tested therapies (bevacizumab (250 μg/mL), aflibercept (250 μg/mL) with/without 3OC12-HSL (200 μM) were immediately applied post-sacrifice in (50 μL) to the corneal surface of each animal for a period of 1 hour, before being washed off using ice-cold PBS. The eyeballs were enucleated, immersed in OCT, and flash-frozen in isopentane cooled by liquid N₂ before being stored at −80° C. for further processing.

Fresh full-thickness porcine skin was obtained from RB Elliot & Son Stud Farm, Calow, immediately post-sacrifice and kept at 4° C. for 60 minutes. 5 cm squares were prepared by scalpel and a central zone of the epidermis delimeted by lipophilic “Pap pen” (Abcam) to locally contain applied liquid. Tested therapy (bevacizumab (250 μg/mL) with/without 3OC12-HSL (200 μM) was applied in PBS to the zone delimited by the Pap pen and the samples kept at 37° for 60 minutes. Skin surface was washed 3× with room-temperature PBS and samples were fixed in 4% paraformaldehyde overnight at 4° C. Fixed samples were sliced in two with a scalpel to expose the central zone of application, immersed in OCT, then plunge-frozen in liquid-N₂-cooled isopentane and stored at −80° C. for further processing.

### Tissue Sectioning

OCT-embedded eyes were cut into 15 μm tissue sections using a cryostat (Leica 3050) and mounted on gelatin-coated slides, at cutting temperatures between −15 and −23° C. Moisture trapped within sections was sublimated on dry ice for 30 minutes in an open box, to prevent the diffusion of agents within the sections, which remained frozen throughout. Slides were then stored at −80° C.

OCT-embedded eyes and skin samples were cut into 15 μm tissue sections using a cryostat (Leica 3050) and mounted on gelatin-coated slides, at cutting temperatures between −15 and −23° C. Moisture trapped within sections was sublimated on dry ice for 30 minutes in an open box, to prevent the diffusion of agents within the sections, which remained frozen throughout. Slides were then stored at −80° C.

### Tissue Immunostaining

50 μL of ice-cold methanol was immediately added to each tissue section for 8 minutes upon removal from the −80° C. freezer to fix and precipitate all proteins in place. All sections were blocked by incubating with a blocking buffer (1% horse serum in PBS) for 30 minutes at room temperature. DyLight 550-Goat anti-Human IgG Fc antibody (1:200) was applied to sections for 1 hour before thorough PBS washing (3x, 15 min) and mounting by anti-fade media for confocal microscopy (Zeiss LSM 880 confocal microscope).

### Results

### Effect of 3OC12-HSL on Drug Permeability Ex-Vivo

The confocal laser scanning microscopy (CLSM) imaging showed, via fluorescence, the successful delivery of clinical formulations of aflibercept and bevacizumab across the corneaa when applied with 3OC12-HSL, and thus into diffusional reach of the anterior ciliary circulation-which directly perfuses the posterior retina.

No fluorescence was detected in the eye sections when bevacizumab and aflibercept were applied without 3OC12-HSL, nor in eye sections treated only with vehicle (HBSS/0.1% DMSO).

### Example E—In Vivo Studies

In vivo studies were carried out to confirm that the effect on epithelial barriers as shown in vitro and ex vivo was also replicated in vivo.

3OC12-HSL was used as an exemplary agent for these studies.

### Hyperglycaemic Induction and Glucose Measurements

A total of 24 male Norway Brown rats (250-300 g) were weighed and given a single intraperitoneal (i.p.) injection of streptozotocin (STZ) 50 mg/kg (Sigma-Aldrich, MO, U.S.A.). Non-diabetic controls were injected with an equivalent volume of saline. In addition to water, a 15% (w/v) sucrose solution was made available in a separate drinking bottle to alleviate the initial hypoglycaemic spike following STZ induction. This volume of sucrose intake was monitored over a 72-hour period. On day 4 animals were anaesthetised with isoflurane (2-5%) and subcutaneously implanted at the nape of the neck with one third of a single Linplant insulin pellet (LinShin, Canada). Blood samples were taken from the tail vein on days 0, 4 and prior to sacrifice (day 28) and blood glucose levels were measured using an Accu-Chek blood glucose monitor. Rats with blood glucose levels of >15 mmol/l and above were deemed hyperglycaemic. STZ-dosed rats that did not become hyperglycaemic on day 4 were re-injected with STZ the following morning and subsequently evaluated for diabetes, as outlined above.

### Topical Eye Treatment Regimen

Animals were given twice daily topical eye drops of 250 μg/ml bevacizumab with/without 3OC12-HSL (200 μM) and control vehicle only (25 μL per eye). The animals were gently restrained for approximately 30 seconds to allow the liquid drop to transform into a gel upon thermal activation with the corneal surface. Animals were checked twice daily for signs of inflammation to the eye and for any evidence of eye or nasal discharge.

### Fundus Fluorescein Angiography (FFA)

Sodium fluorescein salt (Na-Fl; M.W. 376.27) was prepared in sterile PBS to give a final concentration of 10 mg/ml (w/v), 0.2 μm filtered and stored at room temperature and away from direct light until required. Animals were anaesthetized with a combination of ketamine hydrochloride, 37.5 mg/kg (Ketaset®, 100 mg/mL) and medetomidine hydrochloride, 0.25 mg/kg (Sedastart®, 1 mg/mL)) i.p. and transferred to an image cradle fitted with a heat mat. A drop of phenylephrine hydrochloride, 5% and tropicamide, 0.8% were applied to the left eye to dilate the pupil. Viscotears was applied to the cornea of both eyes to prevent dehydration and whiskers were placed out of the field of view using a cotton tip. The Micron IV ophthalmoscope (Phoenix Technology Group Inc.) was advanced towards the cornea and the optic nerve was centred in the field of view, altering the brightness and focus to achieve a crisp image of the main retinal vessels. Once the eye was correctly aligned bright field fundus images of the retina were captured to check for any ocular abnormalities. The FITC filter was selected and a 3-min video footage of the retina was recorded at 15 frames per second, maximum gain. Rats then received a single 250 μl i.p. injection of Na-Fl (10 mg/ml, w/v). Post imaging the animals were recovered with atipamezole hydrochloride (Sedastop®, 5 mg/mL). This was repeated on days 7, 14, 21 and 28 aligning the eye in the same position as captured on Day 0.

### FFA Analysis

Angiograms were imported into Fiji software as a virtual stack. Using the regions of interest tool an area within a main retinal vessel and a second in the interstitium (including unresolved capillaries) were defined using the rectangular selection tool. Na-Fl intensity in the retinal interstitium and a main retinal vessel were measured every 200 frames from 0 to 1800 frames (120 s) and plot against time. Intensity values and time were processed through a macro in Fiji and retinal permeability calculated.

### Laser-Induced Choroidal Neovascularisation

Six to eight week-old female C57BL/6J mice (16-20 g, Charles River, UK) were anaesthetized with an intraperitoneal (i.p.) injection of 50 mg/kg ketamine hydrochloride (Ketaset®, 100 mg/mL) and 0.5 mg/kg medetomidine hydrochloride (Sedastart®, 1 mg/mL). The pupils were dilated with a drop of 5% phenylephrine hydrochloride and 0.8% tropicamide to each eye. Four photocoagulation lesions were delivered with a green Merilas laser (532a, 450 mW, 130 ms, 75 μm) in combination with a Micron IV ophthalmoscope between the retinal vessels in a peripapillary distribution, at a distance of 1-2 disc-diameters from the optic nerve, in each eye. Only laser lesions with a sub-retinal bubble at the time of treatment were included in the study. Immediately following laser photocoagulation the animals received topical drops to both eyes, twice daily (10 μL of 250 μg/mL bevacizumab with/without 3OC12-HSL, 200 μM and control vehicle only)). On days 7 and 14 animals were anaesthetized and pupils dilated as above and given an i.p. injection of 100 μL 10 mg/mL Na-Fl in PBS. The retinae in both eyes were then imaged using the Micron IV ophthalmoscope. Fundus angiograms were imported into Fiji software and lesions were traced by a masked observer and lesion area was quantified over treatment time and compared between treatment groups. Animals were culled on day 14 post imaging and the eyes were enucleated and fixed in 4% paraformaldehyde (w/v), for 1 h.

### Tissue Sectioning

OCT-embedded eyes were cut into 15 μm tissue sections using a cryostat (Leica 3050) and mounted on gelatin-coated slides, at cutting temperatures between −18 and −23° C. Moisture trapped within sections was sublimated on dry ice for 30 minutes in an open box, to prevent the diffusion of agents within the sections, which remained frozen throughout. Slides were then stored at −80° C.

### Tissue Immunostaining

50 μL of ice-cold methanol was immediately added to each tissue section for 8 minutes upon removal from the −80° C. freezer to fix and precipitate all proteins in place. All sections were blocked by incubating with a blocking buffer (1% horse serum in PBS) for 30 minutes at room temperature. DyLight 550-Goat anti-Human IgG Fc antibody (1:200) was applied to sections for 1 hour before thorough PBS washing (3×, 15 min) and mounting by anti-fade media for confocal microscopy (Zeiss LSM 880 confocal microscope).

### Statistical Analysis

Data were gained from three independent replicate experiments. All data, and graphs were formulated with Microsoft Excel (Microsoft Office Software), GraphPad Prism v7/8 (GraphPad Software Inc., CA, U.S.A.), Fiji and Imaris. Data analysis was performed using one-way and two-way ANOVA followed by Bonferroni post-hoc correction.

Unless otherwise stated, all data are shown as mean±SEM. All results were considered statistically significant at p<0.05 (*), p<0.01 (**), p<0.001 (***), p<0.0001 (****).

### 3OC12-HSL Enhances Delivery of Topically Administered Bevacizumab to the Posterior Chamber of Rodent Eyes.

Bevacizumab was formulated in pluronic F127 in the presence and absence of 3OC12-HSL (100 μM) and applied topically to the cornea. Two different rodent strains were chosen to investigate the efficacy of 3OC12-HSL as a drug permeation enhancer in conjunction with Bevacizumab. The latter is currently administered by multiple intravitreal injections to achieve therapeutic concentrations in the posterior segment of the eye and site of disease. Topical delivery of drug molecules to the back of the eye is a major challenge due to the many layers of the eye that the drug is required to permeate, vascular blood-aqueous and blood-retinal barriers, choroidal and conjunctival blood flow, lymphatic clearance, efflux pumps and tear dilution. Drugs that are successful in reaching the posterior segment do so via the cornea, sclera and conjunctival tissues by passive diffusion down the concentration gradient. Therefore permeation adjuncts such as 3OC12-HSL may revolutionise how posterior eye diseases is presently treated.

Confocal imaging of sagittal cryosections of eyes depicted the markedly enhanced permeability of Bevacizumab when co-applied with 3OC12-HSL in comparison with application of Bevacizumab alone. The presence of 3OC12-HSL was found to significantly enhance the delivery of Bevacizumab into the posterior chamber of rat eyes as denoted by an increased expression of Bevacizumab in the confocal images.

To further support the above, in vivo data obtained from a diabetic rat model with increased retinal permeability confirmed a statistically significant reduction (p=0.023) in vascular leak after 28 days, in rats treated topically, twice daily with Bevacizumab plus 3OC12-HSL (0.083±0.044 P_{Fluorescein} μm/s) compared to Bevacizumab alone (0.316±0.078 P_{Fluorescein} μm/s) (FIG. 17D) or vehicle control (1.253±0.253 P_{Fluorescein} μm/s) (FIG. 6C). As previously published vascular leak significantly increased in a time dependent manner in the vehicle onlycontrol group (FIGS. 6A-D).

In addition to the diabetic retinopathy model 3OC12-HSL was trialled as a potential drug permeation enhancer in a laser-induced model of CNV. Treatment with Bevacizumab (250 μg/mL) alone reduced average lesion area at day 7 (2.86±0.47×10⁴) when compared to vehicle control (3.75±0.53). Interestingly Bevacizumab in combination with 3OC12-HSL significantly reduced lesion size at day 7 (0.19±0.18×10⁴, p=0.002) when compared to vehicle control (3.05±0.45×10⁴), confirming the effectiveness of 3OC12-HSL as a drug permeation enhancer in neovascular eye disease (FIG. 7).

### Example F: Further Studies

The results of further Transwell studies are shown in FIGS. 8 and 9.

FIG. 8 shows the results for 12 studied molecules which were investigated in coarse dose-response by FD4 delivery in Transwell culture (Calu-3) to confirm the effect on FITC-dextran (4 kDa) transport. Each molecule was screened at 5 nM, 1 μM, 200 μM levels. FIG. 9 shows the results for 6 further studied molecules which were screened at 200 μM level.

### Discussion and Conclusions

A number of agents have been shown to have benefits as excipient penetration enhancers to increase macromolecular therapeutic transport across in-vitro and ex-vivo epithelial layers through the targeted and reversible modulation of tissue barrier integrity.

Having a resolution phase, i.e. repair, is important, because to be useful in practical and therapeutic terms the disruption of epithelial barriers needs to be temporary rather than there being permanent disruption or damage.

Taken together, the results demonstrate that the agents according to the invention are capable of inducing reversible disruption of epithelial barrier integrity.

The enhanced trans-barrier delivery of both macromolecular (aflibercept, bevacizumab, mepolizumab) and small molecule (doxorubicin) therapeutics has been observed after the application of agents according to the invention, and this has been shown to be due to the opening of tight junctions rather than the result of toxicity.

It is surprising that macromolecular agents (aflibercept, bevacizumab, mepolizumab: ˜150 kDa) as well as small molecules have been shown to be effectively delivered across epithelial barriers by using the present invention. It would not have been expected that molecules of this size could be delivered. The present invention advantageously provides a versatile approach that can be used with a wide range of payload molecules. To be able to effectively transport drugs having a range of sizes by penetrating the epithelial barrier is technically significant and offers significant advantages over intravenous administration.

The in-vitro experiments carried out demonstrated that bevacizumab and aflibercept transport was enhanced significantly across mature ARPE-19 monolayers and across Calu-3 and Caco-2 model epithelial barriers in the presence of agents according to the invention. Co-application of doxorubicin with agents according to the invention enhanced transport significantly in all model epithelial barriers tested.

Enhanced ex vivo delivery across the cornea of aflibercept and bevacizumab, when co-applied with agents according to the invention, paralleled those obtained using in vitro epithelial models. Enhanced delivery of aflibercept and bevacizumab across the corneal epithelium was confirmed by immunostaining of eye sections, and no aflibercept and bevacizumab was detected in contralateral control eyes.

Topical co-application of agents according to the invention with macromolecular therapeutics to the cornea, as well as to model epithelial barriers, enhances the transepithelial delivery of macromolecular therapies. This is advantageous because currently very little drug penetrates into ocular tissue by topical administration and only drugs with a molecular weight of less than 500 Daltons can penetrate.

In conclusion, the agents according to the invention can be used to increase the permeability of in-vitro and ex-vivo epithelial tissue models to macromolecular therapies. By promoting the transport of these crucial modern therapies via the paracellular route, in a non-toxic and reversible manner, a beneficial new route of excipient action is provided, via the specific, non-toxic, and reversible disruption of epithelial tight junctions.

Thus, the claimed agents can be used as efficient penetration enhancers of drugs to improve drug delivery across epithelial tissue barriers, with beneficial implications to patient comfort and expense in clinical medicine.

1. A method of delivering a payload molecule across an epithelial tissue barrier, the method comprising:
applying the payload molecule to the epithelial tissue barrier, and
additionally applying an agent to the epithelial tissue barrier,
wherein the agent is selected from:
(i) a microbial quorum sensing signalling molecule (microbial QSSM) or a derivative or variant thereof, which is capable of disrupting the epithelial tissue barrier function, and which is a compound that comprises a heterocyclic ring portion and an alkane portion comprising an R′ group which is a C1-12 alkyl group, that may be unsubstituted or substituted, wherein any substituent groups that are present are independently selected from hydroxyl, halogen and NR″₂, where each R″ is independently selected from hydrogen and methyl; or
(ii) a carboxylic acid compound of Formula (I), or a pharmaceutically acceptable salt, hydrate or solvate thereof: 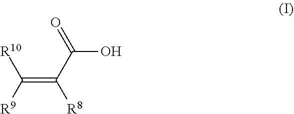
wherein
R⁸ is H or R′; R⁹ is H or R′; and R¹⁰ is H or R′, provided that at least one R′ group is present; wherein each R′ is independently selected from a C1-12 alkyl group, that may be unsubstituted or substituted, wherein any substituent groups that are present are independently selected from hydroxyl, halogen and NR″₂, where each R″ is independently selected from hydrogen and methyl.

2. The method of claim 1, wherein the R′ group is a saturated hydrocarbon chain with no N heteroatoms included within the chain.

3. The method of claim 1 or claim 2, wherein either the R′ group is unbranched or the R′ group is branched and comprises no more than a single C1 branch off the main chain.

4. The method of any one of claims 1-3, wherein if the R′ group is provided as a substituent group on an aromatic or non-aromatic ring, then the substituents at any position ortho to the R′ group are selected from the group consisting of: OH, CH₃, H, R′, and O.

5. The method of any one of claims 1-4, wherein the agent is a microbial QSSM which is a compound of Formula (II), (III), (IV) or (V), or a pharmaceutically acceptable salt, hydrate or solvate thereof: 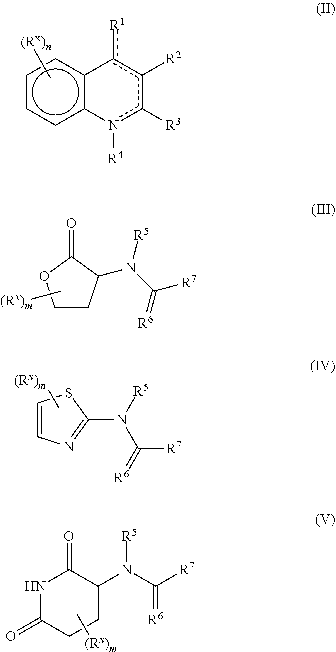
wherein
R¹ is O or OH;
R² is OH, CH₃ or H;
R³ is R′;
R⁴ is H, R′, or O;
R⁵ is H or R′;
R⁶ is O, CH₂, CHR′ or CR′₂;
R⁷ is CH₂C(O) R′ or R′;
wherein each R^{x} is independently selected from halo, C1-12 alkyl, C1-12 alkylhalo, OR^{y}, and NR″₂, and where R^{y} is hydrogen or C1-12 alkyl, and where each R″ is independently selected from hydrogen and methyl; and wherein n is zero or an integer from 1 to 4 and wherein m is zero or an integer from 1 to 2;
wherein each R′ is independently selected from a C1-12 alkyl group, that may be unsubstituted or substituted, wherein any substituent groups that are present are independently selected from hydroxyl, halo and NR″₂, where each R″ is independently selected from hydrogen and methyl.

6. The method of claim 5, wherein the microbial QSSM is a compound of Formula (II) or a pharmaceutically acceptable salt, hydrate or solvate thereof, where one or more of the following applies:
R¹ is O; and/or
R² is OH or H; and/or
R⁴ is H or O.

7. The method of claim 5 or claim 6, wherein the microbial QSSM is a compound of Formula (II) or a pharmaceutically acceptable salt, hydrate or solvate thereof, wherein the fused ring structure comprises a substituted aromatic group where there are one to four (such as one or two) substituent groups R^{x}, each independently selected from:
(i) halo, C1-12 alkyl, C1-12 alkylhalo, and OR^{y}, where R^{y} is hydrogen or C1-12 alkyl; or
(ii) halo (e.g. F), C1-6 alkyl, C1-6 alkylhalo, and OR^{y}, where R^{y} is hydrogen or C1-6 alkyl;
or
(iii) halo (e.g. F), methyl, and hydroxyl.

8. The method of claim 7, wherein the microbial QSSM is a compound of Formula (II) or a pharmaceutically acceptable salt, hydrate or solvate thereof,
wherein the fused ring structure comprises a substituted aromatic group where there are one to four (e.g. one or two) substituent groups R^{x} which are halo groups (e.g. F), optionally wherein the microbial QSSM is the compound 7F-C7PQS or 6F, 7F-C7PQS:

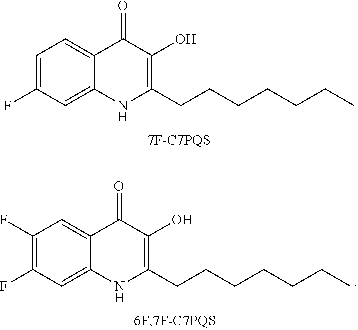 [SMILES: CCCCCCCc1[nH]c2cc(F)ccc2c(=O)c1O ; CCCCCCCc1[nH]c2cc(F)c(F)cc2c(=O)c1O]

9. The method of claim 5 or claim 6, wherein the microbial QSSM is a compound of Formula (II) or a pharmaceutically acceptable salt, hydrate or solvate thereof, wherein n equals 0.

10. The method of claim 9, wherein the microbial QSSM is a compound of Formula (II-A), (II-B), (II-C), (II-D) or (II-E) or a pharmaceutically acceptable salt, hydrate or solvate thereof:

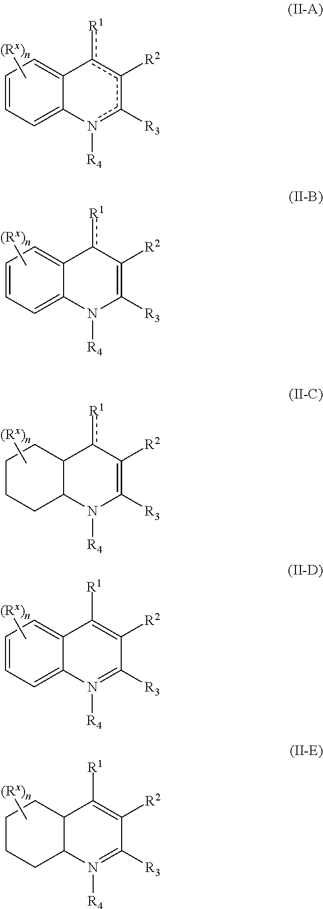

11. The method of claim 10, wherein the microbial QSSM is a compound of Formula (II-B), or a pharmaceutically acceptable salt, hydrate or solvate thereof, where R¹ is O, where R² is OH or H, and where R⁴ is H or O;
optionally wherein the microbial QSSM is a compound selected from: HHQ, NHQ, UHQ C7 PQS, C1 PQS, C9 PQS, C11 PQS, and 7F-C7PQS, or a pharmaceutically acceptable salt, hydrate or solvate thereof:

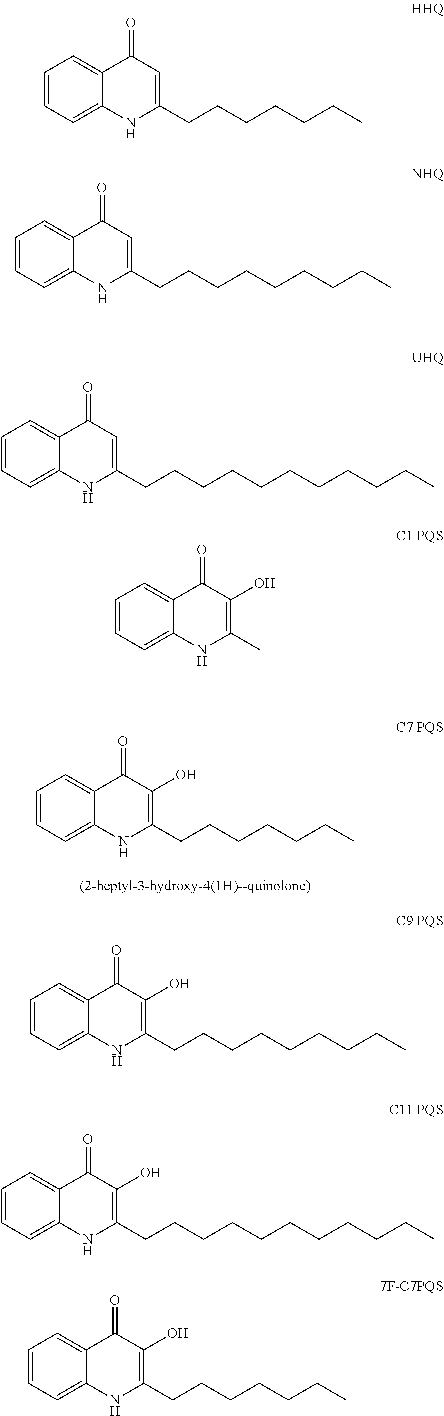

12. The method of any one of claims 5 to 11, wherein the microbial QSSM is a compound of Formula (II) or a pharmaceutically acceptable salt, hydrate or solvate thereof, wherein R⁴ is H or O.

13. The method of claim 12, wherein the microbial QSSM is a compound of Formula (II-F) or (II-G) or a pharmaceutically acceptable salt, hydrate or solvate thereof: 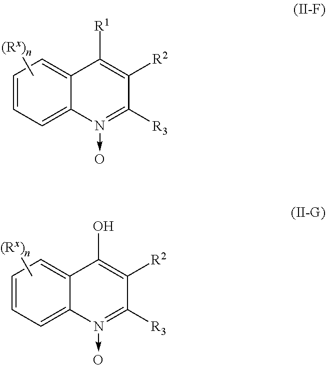
optionally wherein it is the compound C7 HHQ N-oxide or C9 HNQ N-oxide or a pharmaceutically acceptable salt, hydrate or solvate thereof:

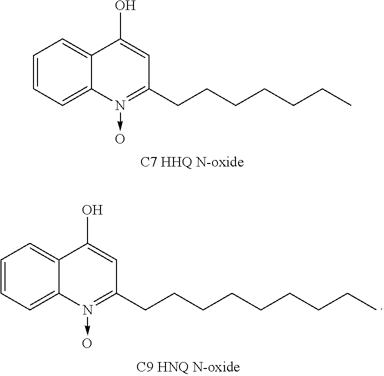 [SMILES: CCCCCCCc1cc(O)c2ccccc2[n+]1[O-] ; CCCCCCCCCc1cc(O)c2ccccc2[n+]1[O-]]

14. The method of claim 5, wherein the microbial QSSM is a compound of Formula (III), (IV) or (V) or a pharmaceutically acceptable salt, hydrate or solvate thereof, where one or both of the following applies:
R⁵ is H; and/or
R⁶ is O or CH₂.

15. The method of claim 5 or claim 14, wherein the microbial QSSM is a compound of Formula (III), (IV) or (V) or a pharmaceutically acceptable salt, hydrate or solvate thereof, wherein there are one or two substituent groups R^{x}, each independently selected from:
(i) halo, C1-12 alkyl, C1-12 alkylhalo, and OR^{y}, where R^{y} is hydrogen or C1-12 alkyl; or
(ii) halo, C1-6 alkyl, C1-6 alkylhalo, and OR^{y}, where R^{y} is hydrogen or C1-6 alkyl;
or
(iii) halo (e.g. F), methyl, and hydroxyl.

16. The method of claim 5 or claim 14, wherein the microbial QSSM is a compound of Formula (III), (IV) or (V) or a pharmaceutically acceptable salt, hydrate or solvate thereof, wherein m equals 0.

17. The method of any one of claims 5 and 14 to 16, wherein the microbial QSSM is a compound of Formula (III), (IV) or (V) or a pharmaceutically acceptable salt, hydrate or solvate thereof, wherein R⁶ is O;
optionally wherein the microbial QSSM is a compound selected from: OdDHL, C4 HSL, OdDAT and OdDG, or a pharmaceutically acceptable salt, hydrate or solvate thereof:

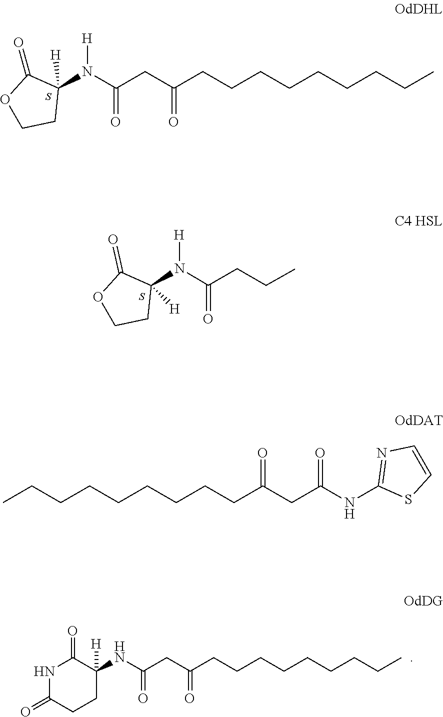

18. The method of any one of the preceding claims, wherein the microbial QSSM (i) is not C7 PQS (2-heptyl-3-hydroxy-4 (1H)-quinolone) and/or (ii) is not 3-oxo-C12 HSL and/or (iii) is not cis-2-decenoic acid.

19. The method of any one of claims 1-3, wherein the agent is a carboxylic acid compound of Formula (I), or a pharmaceutically acceptable salt, hydrate or solvate thereof; wherein R⁸ is H or R′; R⁹ is H or R′; and R¹⁰ is R′.

20. The method of any one of claims 1-3, wherein the agent is a carboxylic acid compound of Formula (I), or a pharmaceutically acceptable salt, hydrate or solvate thereof;
wherein R⁸ is H; R⁹ is H; and R¹⁰ is R′;
optionally wherein the agent is cis-2-decenoic acid.

21. The method according to any one of claims 1-20, wherein the payload molecule and the agent are applied to epithelial tissue barrier simultaneously, sequentially, or separately.

22. The method according to any one of claims 1-21, wherein the payload molecule is a therapeutic or prophylactic agent.

23. The method according to claim 22, wherein the payload molecule comprises:
a) one or more of a peptide, protein, polysaccharide, nucleic acid, nanoparticle, or a small molecule having a molecular weight of less than 900 Da; and/or
b) one or more of a viral particle, a virus-like particle, viral protein, nucleic acid encoding a viral protein, a nanoparticle, an antibody, antibody fragment or mimetic, a protein or peptide complex, cytokine or a toxin; and/or
c) an anti-VEGF antibody, or fragment thereof, or an inhibitor of VEGF and/or the payload molecule comprises an anti-inflammatory; and/or
d) a therapeutic agent that is suitable for treatment or prevention of an eye disorder, a respiratory disorder, a gastrointestinal disorder, reproductive-tract disorder, mucous membrane disorder, a brain disorder, an infection, or a skin disorder; and/or
e) a prophylactic agent, such as a vaccine.

24. The method according to any one of claims 1-23, wherein the epithelial tissue barrier:
a) is in the brain, eye, respiratory system, skin, gastrointestinal tract, urinary tract, male or female reproductive system, olfactory system, or any mucous membrane of a subject; and/or
b) is an internal barrier, such as the blood-brain barrier, blood-testicular barrier, or blood-retinal barrier; and/or
c) is in the respiratory system, the gastrointestinal tract, the urinary tract, or the female reproductive tract.

25. A composition, which may be a pharmaceutically acceptable composition, comprising (a) an agent capable of disrupting the epithelial tissue barrier, and optionally (b) a payload molecule;
wherein the agent capable of disrupting the epithelial tissue barrier is as defined in any one of claims 1-20;
and wherein the payload molecule is optionally as defined in claim 23.

26. The composition according to claim 25, wherein:
a) the composition is in the form of a gel, lotion, cream, ointment, drop, spray, aerosol, or solution; and/or
b) the composition further comprises one or more of water; saline; salt; buffer; demulcent; humectant; viscosity increasing agent; tonicity adjusting agent; cellulose derivatives e.g. carboxymethylcellulose sodium, hydroxyethyl cellulose, hydroxypropyl methylcellulose, or methylcellulose; dextran 70; gelatin; polyols; glycerine; polyethylene glycol e.g. PEG300 or PEG400; polysorbate 80; propylene glycol; polyvinyl alcohol; and povidone (polyvinyl pyrrolidone); poloxamer, such as poloxamer 407; and combinations thereof; and/or
c) the agent capable of disrupting the epithelial tissue barrier is encapsulated or impregnated within a polymer, or is adsorbed to a macromolecular carrier or protein, or is crystallised, lyophilised or manufactured into nanoparticles.

27. The composition as defined in claim 25 or 26 for use as a medicament, wherein the composition comprises a therapeutic or prophylactic payload molecule.

28. The composition for use according to claim 27, wherein the use is in a method of treatment or prevention of an eye disorder, a respiratory disorder, a gastrointestinal disorder, reproductive-tract disorder, mucous membrane disorder, a brain disorder, a microbial or parasitic infection, cancer, or a skin disorder, in a subject.

29. A method of treatment or prevention of an eye disorder, a respiratory disorder, a gastrointestinal disorder, reproductive-tract disorder, mucous membrane disorder, a brain disorder, an infection, cancer, or a skin disorder,
the method comprising administering to a subject a composition as defined in claim 25 or 26, wherein the composition comprises a therapeutic or prophylactic payload molecule.

30. The composition for use in treatment according to claim 28 or the method of treatment according to claim 29, wherein the administration is topical administration to an epithelial barrier.

31. A product comprising a composition as defined in claim 25 or 26, wherein the product is:
a) an eye drop dispenser, eye wash device or contact lens; and/or
b) an aspirator, inhaler, nebuliser, or vape device; and/or
c) a controlled release tablet or capsule suitable for oral administration; and/or
d) a transdermal patch or gel; and/or
e) a vaccine, wherein the vaccine further comprising an antigen or a nucleic acid (such as a viral vector) suitable for expression of an antigen.

32. A kit comprising:
(a) an agent capable of disrupting the epithelial tissue barrier, and
(b) a payload molecule;
wherein the agent capable of disrupting the epithelial tissue barrier is as defined in any one of claims 1-20;
and wherein the payload molecule is optionally as defined in claim 23.

33. Use of an agent capable of disrupting the epithelial tissue barrier to facilitate penetration of a payload molecule through an epithelial tissue barrier,
wherein the agent capable of disrupting the epithelial tissue barrier is as defined in any one of claims 1-20;
and wherein the payload molecule is optionally as defined in claim 23.

34. Use of an agent capable of disrupting the epithelial tissue barrier to facilitate extracellular fluid extraction from a subject, wherein the use comprises the application of the agent capable of disrupting the epithelial tissue barrier to an epithelial tissue barrier and the extraction of extracellular fluid through the epithelial tissue barrier, wherein the agent capable of disrupting the epithelial tissue barrier is as defined in any one of claims 1-20.

35. A method for extracellular fluid extraction from a subject, the method comprising:
application of an agent capable of disrupting the epithelial tissue barrier to an epithelial tissue barrier of the subject; and
extraction of extracellular fluid through the epithelial tissue barrier;
wherein the agent capable of disrupting the epithelial tissue barrier is as defined in any one of claims 1-20.

36. The use according to claim 34 or the method according to claim 35, wherein the extraction of extracellular fluid through the epithelial tissue barrier is carried out by applying a vacuum to the agent treated surface to extract the extracellular fluid.

37. The use according to claim 34 or 36 or the method according to claim 35 or 36, wherein extracted extracellular fluid is used for analysis, such as for electrophysiological measurements and/or biomarker analysis.
